(19) United States
(12) Patent Application Publication
(10) Pub. No.: US 2024/0368630 A1
(43) Pub. Date: Nov. 7, 2024

(54) A NOVEL RNA-PROGRAMMABLE SYSTEM FOR TARGETING POLYNUCLEOTIDES

(71) Applicant: VILNIUS UNIVERSITY, Vilnius (LT)

(72) Inventors: Virginijus SIKSNYS, Vilnius (LT); Tautvydas KARVELIS, Vilnius (LT)

(21) Appl. No.: 18/575,173

(22) PCT Filed: Jul. 2, 2021
(86) PCT No.: PCT/IB2021/055958

§ 371 Date: Dec. 28, 2023

(51) Int. Cl. C12N 15/90; C12N 9/22; C12N 15/11

(52) U.S. Cl. CPC C12N 15/907; C12N 9/22; C12N 15/11; C12N 15/90; C12N 2310/20; C12N 2800/90

(57) ABSTRACT

The present invention concerns a method for cleaving polynucleotides with an effector complex comprising an RNA and a protein comprising a TnpB protein, and related methods and products.

### TECHNICAL FIELD

The present disclosure relates to the field of targeted polynucleotide modification and detection of target sequences in polynucleotides.

### BACKGROUND

Microorganisms have long been a source of interesting and useful tools for genetic engineering, for applications across a wide range of technologies, including in medicine.

In recent years, there has been particular interest in adapting CRISPR-Cas systems, which are derived from bacterial and archaeal adaptive immune systems, for use in DNA modification. In nature CRISPR-Cas systems are highly diverse and have been categorized into two classes, each currently comprising three different types with multiple sub-types (as recently reviewed by Makarova et al., 2020). The most studied of these are a system based on CRISPR-Cas9 (from class 1, type II) (as reviewed by Jiang and Doudna, 2017), and more recently a system based on CRISPR-Cas12 (from class 2, type V) (as described, for example, by Zetsche et al., 2015 and Karvelis et al., 2020). These systems are based around RNA-guided DNA endonucleases arranged as ribonucleoprotein (RNP) complexes, which are capable of introducing site-specific double-stranded breaks into target DNA. In both cases target recognition by the RNP complex requires the presence of a short protospacer adjacent motif (PAM) flanking the target site. Nevertheless, the ability to direct the endonuclease to different targets by changing the RNA sequence (provided a suitable PAM is present), makes these systems highly attractive as a source of tools for DNA modification.

### SUMMARY

In the present application it is surprising shown that TnpB proteins are RNA-binding proteins, which form ribonucleoprotein effector complexes with an RNA molecule. Moreover, it is shown that these effector complexes are capable of cleaving polynucleotides based on binding of a segment of the RNA molecule to a target sequence in a polynucleotide and the subsequent nuclease activity of the TnpB protein in the complex.

TnpB proteins are known in the art as the predicted product of the tnpB gene, which is found in some families of bacterial and archaeal insertion sequences (ISs). Insertion sequences are widespread prokaryotic mobile genetic elements, to which a significant number of eukaryotic DNA transposable elements are related (Hickman et al., 2010). Insertion sequences only contain genes related to transposition and the regulation of transposition. Some families of insertion sequences carry a tnpA gene as well as a tnpB gene. However, while the function of TnpA in transposition is well established, the role of TnpB has not been shown. It has been shown that TnpB is not essential for transposition and the protein is thought to be involved in the negative regulation of transposon excision and insertion (Kersulyte et al., 2000, 2002; Pasternak et al., 2013). It has never previously been shown that TnpB proteins can act as nucleases when bound to RNA, nor that cleavage is targeted by binding between a segment of the RNA and a target site.

The experiments described herein show that TnpB proteins can be used to produce novel RNA-guided effector complexes, in which the TnpB protein can act as a nuclease, and which are functionally distinct from the CRISPR-Cas9 and the CRISPR-Cas12 systems of the prior art. Unlike the CRISPR-Cas systems, there is no CRISPR array associated with the insertion sequences. Rather it has surprisingly been shown that the RNA with which the TnpB protein is associated in nature comes from a part of the insertion sequence.

These effector complexes described herein have significant utility in targeting polynucleotides in vitro, ex vivo or in vivo, and advantageously expand the gene modification toolbox. As well as modifying polynucleotides utilising the nuclease activity of the TnpB protein, the TnpB protein may be mutated to inactivate the nuclease activity allowing the effector complex to be used to block gene expression, or to be used to detect a target sequence, without cleavage of the polynucleotides.

Moreover, the effector complexes described herein, comprising the active or the inactive forms of the TnpB protein can be engineered to carry one or more additional effector molecules to the target site within the polynucleotide. In some examples, the TnpB protein, or the inactivated form thereof, may be comprised in a fusion protein with the one or more effector molecules.

Since TnpB proteins are relatively small in size, they are particularly suitable for delivery to cells, for example, by AAV-based delivery, and use in therapeutic applications. In certain situations where the size of the effector complex is important, the TnpB-based effector complexes of the present invention are advantageous over the larger Cas9 and Cas12 proteins, which are 1000-1500 amino acids in length and 500 to 1500 amino acids in length, respectively.

Accordingly, the present invention provides the following:

In a first aspect the present invention provides a method for cleaving a polynucleotide with an effector complex, wherein the polynucleotide comprises a target sequence, the effector complex comprising:
(a) a protein comprising or consisting of a TnpB protein; and
(b) an RNA comprising:
   (i) a polynucleotide-targeting segment comprising a guide sequence capable of hybridising to the target sequence; and
   (ii) a protein-binding segment that allows the RNA to bind the TnpB protein to form the effector complex,
      wherein the method comprises contacting the polynucleotide with the effector complex and allowing the TnpB protein to cleave the polynucleotide.

In a second aspect the present invention provides an RNA for guiding an effector complex to a target region in a polynucleotide, the RNA comprising:
(i) a polynucleotide-targeting segment comprising a guide sequence capable of hybridising to a target sequence in the target region of the polynucleotide; and
(ii) a protein-binding segment that allows the RNA to bind to a TnpB protein to form the effector complex.

In a third aspect the present invention provides an effector complex for binding to a target region in a polynucleotide, the effector complex comprising a protein and an RNA, wherein the protein comprises or consists of a TnpB protein, and wherein the RNA comprises:
(i) a polynucleotide-targeting segment comprising a guide sequence that is capable of hybridising with a target sequence that is comprised in the target region; and
(ii) a protein-binding segment that binds to the TnpB protein.

In a fourth aspect the present invention provides a fusion protein, wherein the fusion protein comprises a TnpB protein and (i) one or more nuclear localisation signals and/or cell penetrating peptides on an amino or a carboxyl terminal end of the fusion protein, and/or (ii) one or more effector molecules.

In a fifth aspect the present invention provides a mutated TnpB protein comprising a mutation to inactive the nuclease domain of the protein optionally wherein the mutated TnpB protein is the TnpB protein of the fusion protein of invention.

In a sixth aspect the present invention provides DNA encoding the RNA.

In a seventh aspect the present invention provides DNA or RNA encoding the fusion protein.

In an eighth aspect the present invention provides DNA or RNA encoding the mutated TnpB protein.

In a ninth aspect the present invention provides a recombinant expression vector comprising the DNA of the invention.

In a tenth aspect the present invention provides a host cell comprising the recombinant expression vector of the invention or the DNA of the invention.

In an eleventh aspect the present invention provides a composition comprising the RNA of the invention, the effector complex of the invention, the fusion protein of the invention, the mutated TnpB protein of the invention, the DNA of the invention, the recombinant expression vector of the invention or the host cell of invention, and a buffer.

In a twelfth aspect the present invention provides methods for in vivo, ex vivo or in vitro methods for producing the RNA, the effector complex, the fusion protein or the mutated TnpB protein of the invention.

In a thirteenth aspect the present invention provides a system for modifying a target region in a polynucleotide, wherein the target region comprises a target sequence, the system comprising:
a) a protein comprising or consisting of a TnpB protein, or DNA encoding said protein, and
b) an RNA, or DNA encoding the RNA, the RNA comprising:
   (i) a polynucleotide-targeting segment comprising a sequence that is complementary to the target sequence; and
   (ii) a protein-binding segment that binds the TnpB protein.

In a fourteenth aspect the present invention provides the RNA, the effector complex, the mutated TnpB, the fusion protein, DNA encoding the foregoing, or the system, for use as a medicament or for use in a method of diagnosis.

In a fifteenth aspect the present invention provides use of the RNA, the effector complex, the mutated TnpB, the fusion protein, DNA encoding the foregoing, or the system, in an ex vivo or in vitro method of determining the presence of a polynucleotide comprising a target sequence in a sample.

In a sixteenth aspect the present invention provides use of the RNA, the effector complex, the mutated TnpB, the fusion protein, DNA encoding the foregoing, or the system, in an in vivo, ex vivo or in vitro method for modifying a target region of a polynucleotide, wherein the target region comprises a target sequence.

In a seventeenth aspect the present invention provides use of the RNA, the effector complex, the mutated TnpB, the fusion protein, DNA encoding the foregoing, or the system, in an in vivo, ex vivo or in vitro method for genetically modify a cell.

In an eighteenth aspect the present invention provides genetically modified cells for use as a medicament in a subject, wherein the cells are obtained by a method comprising genetically modifying cells obtained from the subject using the system or the effector complex of the invention.

In a nineteenth aspect, the present invention provides a method for modifying, labelling or controlling expression from a target region in a polynucleotide with an effector complex, wherein the target region comprises a target sequence,
wherein the effector complex: (i) is an effector complex of the invention; (ii) comprises a fusion protein and an RNA of the invention; or (iii) comprises a mutated TnpB protein, or a fusion protein comprising the mutated TnpB protein, and an RNA of the invention,
wherein the method comprises contacting the polynucleotide with the effector complex such that the guide sequence of the RNA hybridises to the target sequence, allowing the effector complex to modify or label the target region or control expression from the target region.

### BRIEF DESCRIPTION OF THE DRAWINGS

To assist understanding of the present disclosure and to show how embodiments may be put into effect, reference is made by way of example only to the accompanying drawings in which:

FIG. 1 relates to IS200/IS605 mobile genetic element characterization. FIG. 1A shows a schematic of D. radiodurans ISDra2 locus. The system consists of tnpA and tnpB genes flanked by left and right partially palindromic sequences (LE and RE, respectively). FIG. 1B shows a schematic of TnpA-mediated “peel and paste” transposition mechanism. TnpA dimer mediates transposon excision from host DNA lagging strand during replication forming circular single-stranded DNA intermediate and donor joint. Next, the excised transposon inserts at the acceptor joint into the host lagging DNA strand next to short 5′-TTGAT-3′ (for ISDra2) motif completing the transposition cycle. Transposon excision/insertion sites are marked with triangles. FIG. 1C provides a schematic of the experimental workflow of TnpB complex expression and purification from E. coli cells and bound RNA extraction. FIG. 1D provides the alignment of sRNA sequenced reads to ISDra2 locus. Transposon excision/insertion site are marked with a triangle. The RNA sequences derived from RE clement are the ribonucleotides that may be involved in the hairpin formation and the two ribonucleotides between the hairpin and the triangle, while the last ˜16 nt at the sequenced RNA 3′-ends aligning to transposon flanking DNA—ribonucleotides shown to the right of the triangle.

FIG. 2 relates to TnpB from ISDra2 system purification. FIG. 2A shows an SDS-PAGE gel illustrating elution fractions of proteins bound to HisTrap chelating column prepared from single TnpB expression and purification from E. coli cells. Boxed area represents expected 10×MBP-TnpB protein (95.4 kDa) size bands. FIG. 2B shows SDS-PAGE gel illustrating elution fractions of proteins bound to HisTrap chelating column prepared from TnpB with ISDra2 system expression and purification from E. coli cells. Boxed area represents expected 10×MBP-TnpB protein (95.4 kDa) size bands. FIG. 2C shows an SDS-PAGE gel of pooled fractions containing 10×MBP-TnpB protein. FIG. 2D shows a gel relating to detection and analysis of nucleic acids co-purifying with TnpB protein.

FIG. 3 shows TnpB protein is an RNA-guided dsDNA nuclease. FIG. 3A provides a schematic of the experimental workflow of double-stranded (ds) DNA cleavage activity detection. The reRNA encoding construct contained 16 nt guide sequence. F—forward primer annealing to the ligated adapter. R1 and R2—reverse primers, annealing to plasmid backbone. 7N represents randomized region in plasmid library next to targeted sequence. FIG. 3B shows adapter ligation position determination indicating double strand break (DSB) formation in the targeted sequence. FIG. 3C provides WebLogo representation of motifs identified in 7N randomized region at 20-21 bp F+R1 enriched adapter ligated reads. FIG. 3D provides a schematic showing the experimental workflow of TnpB RNP complex expression and purification. The reRNA encoding construct contained 16 nt guide sequence. FIG. 3E provides gels showing that TnpB RNP complex cleaves supercoiled and linearized target plasmid in vitro and the cleavage is dependent on intact RuvC-like active site. FIG. 3F provides a gel showing that transposase associated motif (TAM) and target complementary to reRNA 3′-end sequence are required for plasmid DNA cleavage. FIG. 3G shows Sanger sequencing of TnpB cleaved plasmid products revealing cleavage position 15-21 bp from the 5′-TAM. Identified cleavage positions are marker with triangles (NTS—non-target strand; TS—target strand).

FIG. 4 shows that TnpB RNP complex cleaves dsDNA in a TAM dependent manner. FIG. 4A shows a schematic of the experimental workflow of double-stranded (ds) DNA cleavage activity detection. The reRNA encoding construct contained 20 nt guide sequence. F—forward primer annealing to ligated adapter. R1 and R2—reverse primers, annealing to plasmid backbone. 7N represents randomized region in plasmid library next to targeted sequence. FIG. 4B shows adapter ligation position determination indicating double strand break (DSB) formation in the targeted sequence. FIG. 4C shows a WebLogo representation of motifs identified in 7N randomized region at 20-21 bp F+R1 enriched adapter ligated reads. FIG. 4D shows a WebLogo representation of motifs identified in 7N randomized region at 20-21 bp F+R1(-TnpB) enriched adapter ligated reads.

FIG. 5 shows TnpB mediated plasmid interference in vivo. FIG. 5A shows a schematic of the experimental workflow of plasmid interference assay in E. coli. The cleavage of target plasmid results in loss of resistance to kanamycin (Kn). The reRNA encoding construct contained 16 nt guide sequence. AmpR—ampicillin/carbenicillin (Ap/Cb) resistance gene, KanR—Kn resistance gene. FIG. 5B shows the results of the transformation experiment. The transformation experiment was serially diluted (10×) and the E. coli transformants grown on the media supplemented with Cb and Kn at 25° C. for 44 h.

FIG. 6 shows TnpB RNP complex purification. FIG. 6A shows a schematic of the experimental workflow of TnpB RNP complex expression and multi-steps purification. The reRNA encoding construct contained 16 nt guide sequence. FIG. 6B shows the results of SDS-PAGE analysis of purified TnpB and TnpB (D191A) RNP complexes. FIG. 6C shows the molecular mass of TnpB and reRNA RNP complex determined by mass-photometry. Obtained molecular mass corresponds to TnpB RNP complex consisting of TnpB protein bound to ˜150 nt reRNA (1:1 molar ratio).

FIG. 7 shows that TnpB nuclease is a novel genome editor. FIG. 7A shows a schematic of the experimental workflow of human cell line (HEK293T) genome editing experiment. FIG. 7B shows indel activity detection in 5 tested 20 bp length targets in human genomic DNA (represented as the mean of 3 replicates, ±standard deviation). Across the x-axis, for each site, bar representing “TnpB (Non-targeting)” is on left hand side, and bar representing “TnpB” is on right hand side. FIG. 7C shows the results of indel profile analysis at EMX1-1 site indicating dominating deletions across cleavage site. Shaded strip on left in the graph represents “TAM” and shaded strip on right represents “Target”.

FIG. 8 shows synthetic dsDNA cleavage by TnpB RNP complex. FIG. 8A provides a gel showing purified TnpB RNP complex cleaves dsDNA substrates containing a target (represented in green color), which is the sequence CTCAGGGAACCGCGGG (SEQ ID NO: 17) (3′→5′) on the TS (target strand), and the TAM (red color), which is represented by the sequence TTGAT (5′→3′) on the NTS (non-target strand), generating a staggered cleavage pattern. NTS and TS represent non-target and target strand, respectively. D—TnpB (D191A) RNP complex incubated with DNA substrate for 60 min. FIG. 8B provides a gel showing purified TnpB RNP complex does not cleave dsDNA substrates containing a target in the absence of the double-stranded TAM. D—TnpB (D191A) RNP complex incubated with DNA substrate for 60 min.

FIG. 9 shows synthetic ssDNA cleavage by TnpB RNP complex. FIGS. 9A and 9B—gels showing purified TnpB RNP complex cleaves ssDNA substrates containing a sequence complementary to the reRNA target sequences. NTS and TS represent non-target and target strand, respectively. D—TnpB (D191A) RNP complex incubated with DNA substrate for 60 min.

FIG. 10 shows the results of TnpB cleavage conditions testing in vitro. FIG. 10A shows the results of an assay to determine TnpB RNP plasmid DNA cleavage at varying temperature. The products were analyzed after 15 min incubation of plasmid DNA with TnpB RNP complex. FIG. 10B shows the results of an assay to determine TnpB RNP plasmid DNA cleavage at varying NaCl concentration. The products were analyzed after 15 min incubation of plasmid DNA with TnpB RNP complex.

FIG. 11 shows TnpB mediated plasmid interference in vivo. FIG. 11A provides a schematic of the experimental workflow of plasmid interference assay in E. coli. The cleavage of target plasmid results in loss of resistance to kanamycin (Kn). The reRNA encoding construct contained 16 nt guide sequence. AmpR—ampicillin/carbenicillin (Ap/Cb) resistance gene, KanR—Kn resistance gene. FIG. 11B shows the results of where the transformation experiments were serially diluted (10×) and the E. coli transformants grown on the media supplemented with Cb and Kn at 25-37° C.

FIG. 12 provides an alignment of the RuvC I, RuvC II and RuvC III motifs of TnpB proteins from different insertion sequences. Sequences of motifs are taken from: ISDra2 (IS605 family) TnpB protein (SEQ ID NO: 1); ISHp608 (IS605 family) TnpB protein (SEQ ID NO: 2); IS605 (IS605 family) TnpB protein (SEQ ID NO: 3); IS606 (IS605 family) TnpB protein (SEQ ID NO: 4); IS609 (IS605 family) TnpB protein (SEQ ID NO: 5); IS1341 (IS1341 family) TnpB protein (SEQ ID NO: 6); ISC1316 (IS1341 family) TnpB protein (SEQ ID NO: 7); IS891 (IS1341 family) TnpB protein (SEQ ID NO: 8); ISEc42 (IS1341 family) TnpB protein (SEQ ID NO: 9); ISTel3 (IS1341 family) TnpB protein (SEQ ID NO: 10); IS607 (IS607 family) TnpB protein (SEQ ID NO: 11); ISTsi1 (IS607 family) TnpB protein (SEQ ID NO: 12); IS1535 (IS607 family) TnpB protein (SEQ ID NO: 13); ISBlo12 (IS607 family) TnpB protein (SEQ ID NO: 14); and ISC1926 (IS607 family) TnpB protein (SEQ ID NO: 15). The alignment shows that the active site residues (D - - - E - - - D—which are boxed) are conserved across the TnpB family.

FIG. 13 provides a schematic of the nuclease activity of the RNA-guided ribonucleoprotein complex of the present disclosure. The ribonucleoprotein complex (comprising a TnpB protein and an RNA) recognises the double-stranded TAM sequence (which is located, referring to the non-target strand, 5′ of the target sequence) and the guide sequence of the RNA in the ribonucleoprotein complex binds the target sequence of the target strand (TS) of the polynucleotide, leading to cleavage of the target strand (TS) and the non-target strand (NTS) by the RuvC-like domain of the TnpB protein.

### DETAILED DESCRIPTION

The present inventors have identified a novel RNA-guided ribonucleoprotein (also referred to herein as “an effector complex”) that functions in a manner that is similar to, but distinct from, Cas9 and Cas12 DNA endonucleases. Accordingly, the present disclosure relates in particular to these effector complexes, methods involving their use for cleaving or modifying a polynucleotide in vitro, ex vivo and in vivo (prokaryotic and eukaryotic cells), and systems for their delivery to target cells.

### The TnpB Protein

The protein of the disclosure is a protein that comprises, consists essentially of or consists of a TnpB protein. In particular, where the protein “comprises” a TnpB protein, further amino acids may be present in the protein. This is described further below and includes fusion proteins of TnpB with one or more additional effector proteins. Where the protein “consists essentially of” the TnpB protein, further amino acids or protein sequences may be present in the protein that do not materially affecting the essential characteristics of the TnpB protein, i.e. its ability to bind to the RNA so as to form an effector complex described herein (which may have the ability to act as a RNA-programmable nuclease, where the TnpB protein retains its nuclease activity, or have the ability to act as a RNA-programmable carrier or RNA-programmable polynucleotide blocker where the TnpB in the effector complex is an inactive/mutant TnpB protein that has had its nuclease activity inactivated as described further below). Where the protein “consists” of the TnpB protein, no further amino acids are present.

TnpB proteins are the proteins encoded by the tnpB gene from insertion sequences (IS), or sequence variants of these TnpB proteins that retain the ability to form the effector complex described herein. In particular, in an example of the disclosure the TnpB protein has an amino acid sequence of a protein obtained from a tnpB gene of a mobile genetic clement in the IS200/IS605 or the IS607 families, or a sequence variant thereof. In a preferred example, the TnpB protein has an amino acid sequence of a protein obtained from a tnpB gene of a mobile genetic element from the IS200/IS605 family, or a sequence variant thereof. More particularly the TnpB protein may have an amino acid sequence of a protein obtained from a tnpB gene of a mobile genetic element from the IS200/IS605 family found in the Deinococcus family of bacteria, or a sequence variant thereof. In one example, the TnpB protein has the amino acid sequence of a TnpB protein obtained from the tnpB gene of ISDra2 (an insertion sequence IS200/IS605 from Deinococcus radiodurans), or a sequence variant thereof.

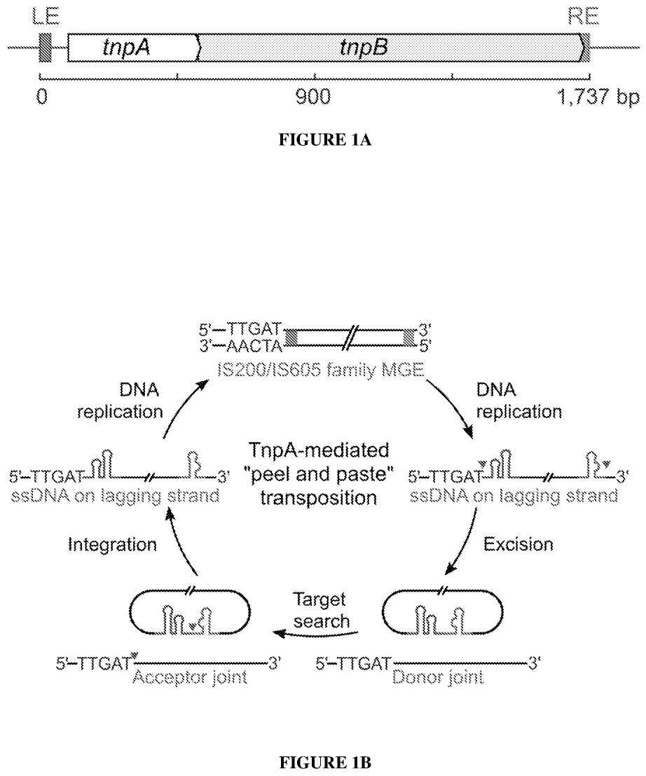

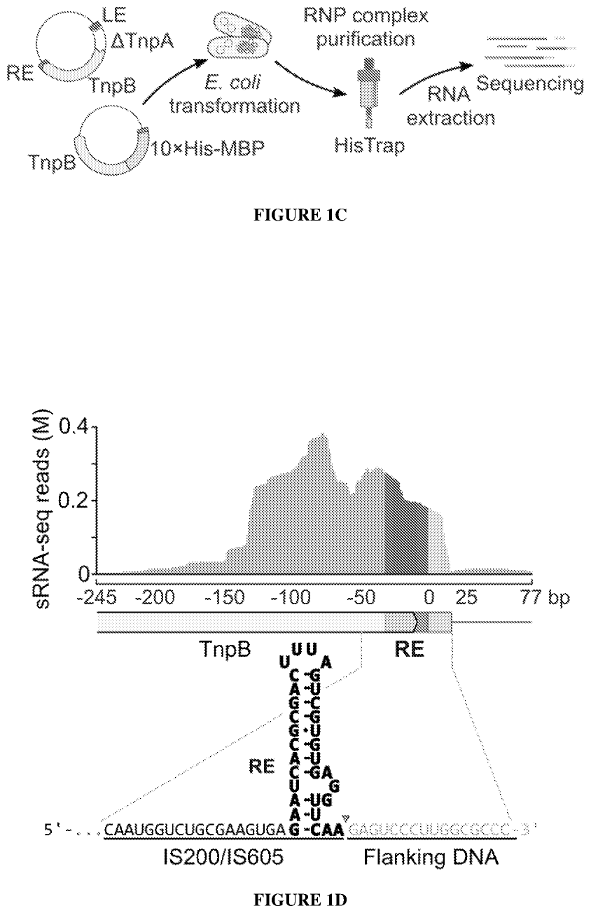

As described above, insertion sequences are simple widespread mobile genetic elements (MGEs) that only contain genes related to transposition and the regulation of transposition. Insertion sequences are classified in the art into different families as described in Siguier et al., 2006 and Siguier et al., 2014, and shown in ISfinder, a database that provides a list of insertion sequences isolated from bacteria and archaea (https://isfinder.biotoul.fr/). While the sequences of these insertion sequences can be diverse, transposable elements of the IS200/IS605 family are identified as those carrying subterminal palindromic elements (LE and RE) at the ends of the MGE and tnpA and tnpB genes in different configurations, or stand-alone tnpA or tnpB genes. In particular, the IS200/IS605 family can be further classified into IS200 (which carry a tnpA gene only), IS200/IS605 (which is sometimes also referred to as IS605 and which carry tnpA and tnpB genes e.g., IS608 from Helicobacter pylori, and ISDra2 of Deinococcus radiodurans, (the arrangement of this element is shown in FIG. 1D)), and IS1341 (which carry a tnpB gene only). IS607 MGEs are identified as those that encode both tnpA and tnpB genes, the coding sequences of which are sometimes overlapping. The ends of these elements may also be associated with inverted repeat sequences, which are often imperfect, and/or secondary RNA structures.

The TnpB proteins comprise an RNA-binding segment and an RuvC-like nuclease domain, that together enable the TnpB protein to form the effector complex described herein which has nuclease activity against a target region (which comprises a target site to which the guide sequence of the RNA binds) in a polynucleotide. In particular, as demonstrated herein the RuvC-like domain is responsible for the nuclease activity of the TnpB protein.

RuvC itself is a dimeric bacterial endonuclease that requires divalent metal ions for activity, and which resolves Holliday junctions in bacteria. RuvC-like domains (comprising RuvC-I, RuvC-II and RuvC-III motifs, optionally with a Zn finger between the RuvC-II and RuvC-III motifs) are known in the art and are recognised as being responsible for cleavage of one DNA strand by the Cas9 protein, and the double-stranded nuclease activity of the Cas12 proteins (see for example, Shmakov et al., 2017, Makarova et al., 2015, and Makarova et al., 2020). Like the RuvC protein, the RuvC-like domain of TnpB normally requires divalent metal ions for activity.

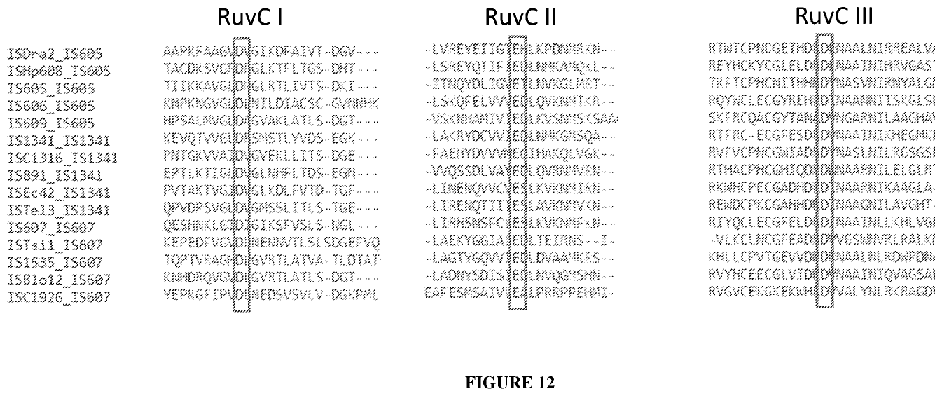

FIG. 12 provides an alignment of the RuvC-I, RuvC-II and RuvC-III motifs of TnpB proteins from different insertion sequences. (Insertion sequence name and family are shown on the left-hand side.) The alignment shows the conserved D - - - E - - - D amino acids in motifs I, II and III, respectively (boxed amino acids in FIG. 12), which are involved in the RuvC active site. These amino acids can be identified within TnpB proteins using sequence alignment tools e.g. Clustal Omega sequence alignment program (https://www.cbi.ac.uk/Tools/msa/clustalo/) (Madeira et al., 2019).

The polynucleotide comprising the target sequence against which the TnpB protein has nuclease activity may be double-stranded DNA, or a single stranded DNA. In particular, the TnpB protein has nuclease activity against double-stranded DNA, and accordingly the effector complex comprising the TnpB protein has particular utility in genome editing.

The RNA-binding segment of the TnpB protein comprises a sequence that interacts with the RNA to form the effector complex. As shown in the experiments reported herein, the present inventors have found that expression of the tnpB gene fused to the sequence encoding a maltose binding protein alone in E. coli and subsequent affinity chromatography revealed low yields of intact TnpB protein. However, co-expression with the RNA resulted in higher yields of the TnpB protein.

Without wishing to be bound by theory the present inventors consider that the interaction of the RNA-binding segment of the TnpB protein with the RNA acts to stabilise the TnpB protein.

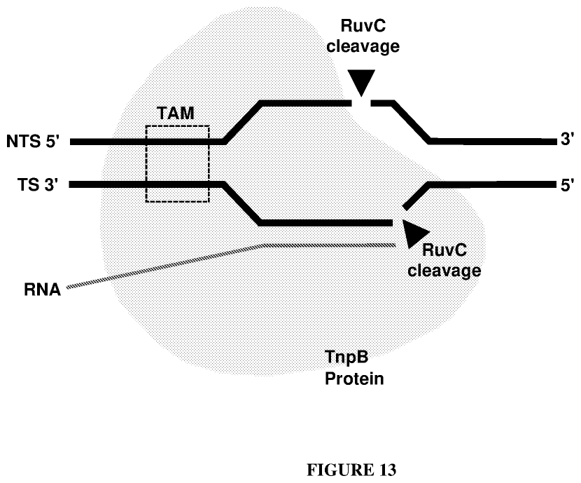

In order to allow the TnpB to cleave double-stranded DNA the polynucleotide should comprise a TnpB-associated sequence motif 5′ of the target sequence (on the non-target strand—as shown in FIG. 13). This TnpB-associated sequence motif is also referred to herein as a Transposon Associated Motif or TAM. In particular, without wishing to be bound by theory, the present inventors consider that effector complex cleavage of the DNA molecule requires the presence of the TnpB-associated sequence motif in a manner similar to the requirement of Cas9 and Cas12 effector proteins for PAM, and that the TAM is recognised by the effector complex as a double-stranded motif (since as shown by the examples herein, its presence is not required for cleavage of single-stranded DNA by the effector complex). It is expected that the sequence of the TAM may vary between different TnpB proteins. The sequence of TAM for a particular TnpB protein can be determined using the PAM (protospacer adjacent motif) identification assay developed previously for Cas9 and Cas12 nuclease (Karvelis et al., 2015, 2019) (see also Example 2).

The TnB-associated sequence motif in the polynucleotide may a T-rich motif, and may be TTGAT. In particular, preferably the TnpB-associated sequence is TTGAT and the TnpB protein is derived from the ISDra2 family, and more preferably comprises or consists of the amino acid sequence of SEQ ID NO: 1 or a sequence variant thereof.

The TnpB protein may be the product of a tnpB gene found in an insertion sequence, or a sequence variant thereof, i.e. be derived therefrom. The TnpB sequence variants retain an RNA-binding segment and an RuvC-like nuclease domain, that together enable the TnpB protein to form the effector complex described herein which has nuclease activity against a target region (which comprises a target site to which the RNA binds) in a polynucleotide. Where the effector complex is for targeting a polynucleotide that is a double-stranded DNA, the TnpB protein variant also needs to retain the ability to recognise the TnpB-associated motif in the target region of the polynucleotide.

Sequence variants may have at least 85%, at least 90%, at least 95%, at least 98%, at least 99% sequence identity to TnpB proteins produced from the tnpB genes from the IS families indicated above. Alternatively, variants may have at least 85%, at least 90%, at least 95%, at least 98%, at least 99% sequence similarity to TnpB proteins (in particular as determined by BLAST).

Sequence variations may be made based on established conserved amino acid changes. In addition, methods described in the art that have been used to increase the specificity and activity of Cas9 and Cas12 proteins may also be utilised to create TnpB variants, in particular with decreased off-target nuclease activity. One example is a directed evolution approach.

The TnpB protein may be between 300 and 600 amino acids in length, and optionally 350 to 550 amino acids in length, further optionally between 350 and 450 amino acids in length.

In one example, the TnpB protein may be the TnpB protein from the tnpB gene of ISDra2 (an insertion sequence IS200/IS605 from Deinococcus radiodurans), which is a 408 amino acid sequence, (see https://isfinder.biotoul.fr/scripts/ficheIS.php?name=ISDra2 and NCBI Accession No. AE000513) having the amino acid sequence SEQ ID NO: 1, or a TnpB protein with an amino acid sequence having at least 85% sequence identity to SEQ ID NO: 1, or at least 90%, at least 95%, at least 98%, at least 99% sequence identity therewith.

(SEQ ID NO: 1)
MIRNKAFVVRLYPNAAQTELINRTLGSARFVYNHFLARRIAAYKESGKG
LTYGQTSSELTLLKQAEETSWLSEVDKFALQNSLKNLETAYKNFFRTVK
QSGKKVGFPRFRKKRTGESYRTQFTNNNIQIGEGRLKLPKLGWVKTKGQ
QDIQGKILNVTVRRIHEGHYEASVLCEVEIPYLPAAPKFAAGVDVGIKD
FAIVTDGVRFKHEQNPKYYRSTLKRLRKAQQTLSRRKKGSARYGKAKTK
LARIHKRIVNKRQDFLHKLTTSLVREYEIIGTEHLKPDNMRKNRRLALS
ISDAGWGEFIRQLEYKAAWYGRLVSKVSPYFPSSQLCHDCGFKNPEVKN
LAVRTWTCPNCGETHDRDENAALNIRREALVAAGISDTLNAHGGYVRPA
SAGNGLRSENHATLVV

In further examples, the TnpB protein may be one of the following, or a sequence variant having at least 85%, at least 90%, at least 95%, at least 98%, or at least 99% sequence identity therewith: ISHp608 (IS605 family) TnpB protein. Length: 383; From NCBI Accession No. AF357224 IS finder database entry: https://isfinder.biotoul.fr/scripts/ficheIS.php?name=ISHp608

(SEQ ID NO: 2)
MLITYKQKLYKNDKNRRIDTLLRRYGALYNHCIALHKRYYRLFKKYLKL
YDLQKHITKLKKTHRYAFLKTLGSQTMQDLTERIDKAFKKFFNKKAKLP
RFKKVANYKSFTFKSKIDKKTGLNKGVGFAIKDNVVSFNGYSYKFIKTY
AFIGKVKTLTIKRDNTGDYFLCLVCELENHPNKQTACDKSVGFDFGLKT
FLTGSDHTKIESPLSFSKYLPLIKRLSKNLSKKVKGSNNFKKAKKKLTQ
LHQKIKYLRTDFFHKLALKLSREYQTIFIEDLNMKAMQKLWGRKVSDLA
FSEFVKILENKANVVKIDRFYPSSKTCSNCLFVNEEINKDFRKIGKTDK
EREYHCKYCGLELDRDLNAAINIHRVGASTLGVEFVRPTC

IS605 (IS605 family) TnpB protein. Length: 427; From NCBI Accession No. HPU60177
IS finder database entry: https://isfinder.biotoul.fr/scripts/ficheIS.php?name=IS605
(SEQ ID NO: 3)
MLNAIKFRIYPNAQQKELISKHFGCSRVVYNYFLDYRQKQYAKGIKETYFTMQKVLTQI
KHQEKYHYLNECNSQSLQMALRQLVSAYDNFFSKRARYPKFKSKKNAKQSFAIPQNIEI
KTETQTIALPKFKEGIKAKLHRELPKDSVIKQAFISCIADQYFCSISYETKEPIPKPTIIKKA
VGLDMGLRTLIVTSDKIEYPHIRFYQKLEKKLTKAQRRLSKKVKGSNNRKKQAKKVAR
LHLACSNTRDDYLHKISNEITNQYDLIGVETLNVKGLMRTYHSKSLANASWGKFLTML
KYKAQRKAKTLLGIDRFFPSSQLCSYCGFNTGKKHENITKFTCPHCNITHHRDYNASVNI
RNYALGMLDDRHKIKIDKSRVGIIRTDYAHYTDERIKACGASSNGVISKYGNILDLASYG
AMKQEKAQSL
IS606 (IS605 family) TnpB protein. Length: 442. From NCBI Accession No. U95957
IS finder database entry: https://isfinder.biotoul.fr/scripts/ficheIS.php?name=IS606
(SEQ ID NO: 4)
MKVNKGFKFRLYPTKEQQDKLQRCFFVYNQAYNIGLNLLQEQYETNKDSPPKERKWK
KSSELDKAIKHHLNARGLSFSSVIAQQSRMNVERALKDAFKVKDRGFPKFKNSKSAKQS
FSWNNQGFSIKDSDEERFKIFTLMKMPLMMRMHRDFPPHSKVKQIVISWSHRKYFVSFC
VEYEQDITPIKNPKNGVGLDLNILDIACSCGVNNHKKLTDFKQYPTDMKELLGIEIDEEL
DTKRLIPTYSKLYSLKKYSKKFKRLQRKQSRRVLKSKQNKTKLGGNFYKTQKKLNQAF
DKSSHQKTDRYHKITSELSKQFELVVVEDLQVKNMTKRAKLKNVKQKSGLNQSILNTSF
YQIISFLDYKQQHNGKLLVKVPPQYTSKTCHCCGNINHKLKLNHRQYWCLECGYREHR
DINAANNIISKGLSLFGVGNIHADFKEQSLSC
IS609 (IS605 family) TnpB protein. Length: 402; From NCBI Accession No. BA000007
IS finder database entry: https://isfinder.biotoul.fr/scripts/ficheIS.php?name=IS609
(SEQ ID NO: 5)
MKRLQAFKFQLRPGGQQEREMRRFAGACRFVFNRALALQNENHEAGNKYIPYGKMAS
WLVEWKNATETQWLKDAPSQPLQQSLKDLERAYKNFFRKRAAFPRFKKRGQNDAFRY
PQGVKLDQENSRIFLPKLGWMRYRNSRQVTGVVKNVTASQSCGKWYISIQTENEVSTP
VHPSALMVGLDAGVAKLATLSDGTVFGPVNSFQKNQKTLARLQRQLSRKVKFSNNWQ
KQKRKIQRLHSCIANICRDYLHKVTTTVSKNHAMIVIEDLKVSNMSKSAAGTVSQPGRN
VRAKSGLNRSILDQGWYEMRRQLEYKQLWRGGQVLAVPPAYTSQRCACCGHTAKENR
LSQSKFRCQACGYTANADVNGARNILAAGHAVLACGEMVQSGRPLKQEPTEMIQATA
IS1341 (IS1341 family) TnpB protein. Length: 369; From NCBI Accession No. D38778
IS finder database entry: https://isfinder.biotoul.fr/scripts/ficheIS.php?name=IS1341
(SEQ ID NO: 6)
MANKAYQFRLYPTKEQEQLLAKTFGCVRFVYNKMLEERIQMFEKFKDDQESLKQQTCP
TPAKYKKEFPWLKEVDSLALANAQLNLQKAFQHFFSGRAGFPKFKNRKAKQSYTTNM
VNGNIKLSDGYIKLPKLKWIKLKQHREIPAHHIIKSCTITKTKTGKYYISILTEYEHQPAPK
EVQTVVGLDFSMSTLYVDSEGKRANYPRFYRKALETLAKEQRKWSRKKKGSNRWHKQ
RLKVAKLHEKIANQRKDFLHKESHKLAKRYDCVVIEDLNMKGMSQALHFGQGVHDNG
WGMFTTFLQYKLVEQGKKLIKIDKWFPSSKTCSCCGRVKESLSLSERTFRCECGFESDRD
VNAAINIKHEGMKRLAIV
ISC1316 (IS1341 family) TnpB protein. Length: 393; From NCBI Accession No. NC_002754
IS finder database entry: https://isfinder.biotoul.fr/scripts/ficheIS.php?name=ISC1316
(SEQ ID NO: 7)
MPTLGFRFRAYTDEQTLRALKAQLKLTCEIYNTLRWADIYFYQRDGKGLTQTELRQLAL
DLRKQDDEYKQLYSQVVQQVADRYSEAKKRFFEGLARFPKEKKPHKYYSLVYTQSGW
KILHVREIRKGKKNKKKLITLKLSNLGTFKVIVHRDFPLDKVKRVVVKLTRSERIYITFVV
DHEFPKLPNTGKVVAIDVGVEKLLITSDGEYFPNLRPYEKALWKVKHIHRELSRKKFLS
NNWFKAKVKLARAYEHLKNLRTDLYMKLGKWFAEHYDVVVMEGIHAKQLVGKSLRS
LRRRLSDVGFGELRGVLKYQLEKYGKKLILVNPAYTSKTCARCGYVKNDLSLSDRVFV
CPNCGWIADRDYNASLNILRGSGSERPLVWSSALYQYSGKVGL
IS891 (IS1341 family) TnpB protein. Length: 401; From NCBI Accession No. M24855
IS finder database entry: https://isfinder.biotoul.fr/scripts/ficheIS.php?name=IS891
(SEQ ID NO: 8)
MLVFETKLEGTNEQYQLLMRRLKLLVLSNACLRTWIGQPNIGRYDLSAYCAVLLPMKT
FRSLPNSTLWLDKLLLKERGVQLLGFLTIASKTKPGRKVIHALKKNRRMGVLSIKLAAG
SLVVTVAYVTFSDGFKAGTFKLWGTRDLHFYQLKQFKRVRVVRRADGYYAQFCIDQE
RVERREPTLKTIGLDVGLNHFLTDSEGNTVENPRHLRKSEKSLKRLQRRLSKTKKGSNN
RVKARNRLSRKHLKVSRQRKDFAVKLARCVVQSSDLVAYEDLQVRNMVRNRHLAKSI
SDAAWTQFRQWVEYFGKVFGVVTVAVPPHHTSQNCSNCGEVVKKSLSTRTHACPHCG
HIQDRDWNAARNILELGLRTVGHTGSQVSGDIDLCLGEVTPPNKSSRGKRKPKK
ISEc42 (IS1341 family) TnpB protein. Length: 376; From NCBI Accession No. NC_004431
IS finder database entry: https://isfinder.biotoul.fr/scripts/ficheIS.php?name=ISEc42
(SEQ ID NO: 9)
MKRAYKYRFYPTTEQAELLAQTFGCVRFVYNSILRWRTDAYYERKEKIGYLQANARLT
ALKKEPEYIWLNDVSCVPLQQSLRHQQAAFANFFAGRAAYPAFKSKRHKQVAEFTASA
FKHRDGELYIAKSKSPLDVRWSRELPSAPSTVTISRDSAGRYFVSCLCEFEPVSMPVTAK
TVGIDVGLKDLFVTDTGFKTDNPRHTAKYAKRLTLLQRRLSRKQKGSRNRIKARLKVA
RLHAKIADCRMDNLHKLSRKLINENQVVCVESLKVKNMIRNPKLSKAIADAGWSELVR
QLQYKGKWAGRSVVAIDQYLPSSKCCSCCGFTMQKMPLNVRKWHCPECGADHDRDIN
AARNIKAAGLAVLAHGEPVNPESQHAA
ISTel3 (IS1341 family) TnpB protein. Length: 393; From NCBI Accession No. NC_004113
IS finder database entry: https://isfinder.biotoul.fr/scripts/ficheIS.php?name=ISTel3
(SEQ ID NO: 10)
MRGVEKAFSYRFYPTTEQESLLRKTLGCVRLVYNRALAARTEAWYERKERLDYVQTSA
LLTQWKKQDDLQFLNEVSCVPLQQALRHLQSAFTNFFAGRAKYPNFKKKRNGGSAEFT
KSAFRWKDGKVFLAKCNEPLNIPWSRRLPDGVEPSTVTIRLNPAGQWYISLRFDDPRELT
LQPVDPSVGLDVGMSSLITLSTGEKIANPKHENRYYKRLRKAQRSLSRKQKGSRNWDK
ARLKVAKIHQKISDSRKDHLHQLTTRLIRENQTIIIESLAVKNMVKNRQLARSISDAGWG
ELVRQLEYKAQWYGRTLVKIDRWFPSSKRCGQCGHIVEWLPLSVREWDCPKCGAHHD
RDINAAGNILAVGHTVTVCGAGVRPDRHTSGGQLRRNRKSQK
IS607 (IS607 family) TnpB protein. Length: 419; From NCBI Accession No. AF189015
IS finder database entry: https://isfinder.biotoul.fr/scripts/ficheIS.php?name=IS607
(SEQ ID NO: 11)
MSAISITHKIALKPNNKHITYFKKAFGCARFAYNWGLAKWKENYQLGIKTSHLQLKKEF
NALKKSQFNFVYEVTKYATQQPFIHLNLAFNKFFRDLEKGLVSYPKFKKKREFQGSFYI
GGDQIKIIQTANTDYLKIPNLPPIKLTEKLRFQGKIHNATITQKGDHFYVSISCDIDESEYK
RTHKLQESHNKLGIDIGIKSFVSLSNGLNIYAPKPLDKLTRKLVRISRQLSKKIHPKTKGD
KTRKSNNYLKHSKKLTHLHEKIANIRLDFLHKLTSSLIRHSNSFCLESLKVKNMFKNHRL
AKSLSDISMSVENTLLEYKAKYSNKEILRADTYYPSSKTCSNCQKVKQDLKLKDRIYQC
LECGFELDRDINAAINLLKHLVGRVTAEFTPMDLTALLNDLSNNRLATSKVELGIQQKS
ISTsi1 (IS607 family) TnpB protein. Length: 393; From NCBI Accession No. NC_012883
IS finder database entry: https://isfinder.biotoul.fr/scripts/ficheIS.php?name=ISTsi1
(SEQ ID NO: 12)
MPSETIKLASKFKLKETPEGLNELFSTYRDIVNFLITHAFENNITSFYRLKKEIYKSLRKEY
PELPSHYIYTACQMAASIYKSYRKRKRRGKASGRPVFKKEAIMLDDHLFKLDLEKGIIKL
STPNGRITLKFYPAKHHEKFKNWKVGQAWLVRTPKGVFINVVFSKEVEVKEPEDFVGV
DLNENNVTLSLSDGEFVQIITHEKEIRTGYFVKRRKIQKKVKVGKKRQELLEKYGERER
NRLNDLYHKLANKIVELAEKYGGIALEDLTEIRNSIRYSAEMNGRLHRWSFRKLQSIIEY
KAKLKGVEVVFVDPAYTSSLCPVCGEKLSPNGHRVLKCLNCGFEADRDVVGSWNVRL
RALKMWGVSVPPESPPMKMGGGKASRGDVYELYTNYG
IS1535 (IS607 family) TnpB protein. Length: 550; From NCBI Accession No. Z95210
IS finder database entry: https://isfinder.biotoul.fr/scripts/ficheIS.php?name=IS1535
(SEQ ID NO: 13)
MIVRMRSCAQAAKVAEATGGVQLAGKPKPDGTPTFSRYVEIGVDFEAHRPVVESVSVL
FELYDGDANSYAATGGPGAQLPSGWMVTAAKFEVEWPADPQRAGLVRSHFGARRKAF
NWGLAQVKADLDAKAADPAHESVDWDLKSLRWAWNRAKDDVAPWWAENSKECYSS
GLADLAQGLANWKAGKNGTRKGRRVGFPRFKSGRRDPGRVRFTTGTMRIEDDRRTITV
PVIGPLRAKENTRRVQRHLVSGRAQILNMTLSQRWGRLFVAVCYALRTPTTRSPLTQPT
VRAGMDLGVRTLATVATLDTATGEQTIIEYPNPAPLKATLVARRRAGRELSRRIPGSHG
HRAVKAKLARLDRRCVHLRREAAHQLTTELAGTYGQVVIEDLDVAAMKRSMRRRAFR
RSVSDAAMGLVAPQLAYKTAKCSGVLTVADRWFASSQIHHGCTSPDGTPCRLQGKGRI
DKHLLCPVTGEVVDRDRNAALNLRDWPDNASRGPVGTTAPSAPGPTTTVGTGHGADT
GSSGAGGASVRPRPRRAGRGEAKTQTPQGDAA
ISBlo12 (IS607 family) TnpB protein. Length: 440; From NCBI Accession No. NC_004307
IS finder database entry: https://isfinder.biotoul.fr/scripts/ficheIS.php?name=ISBlo12
(SEQ ID NO: 14)
MSAYEAVRIRLDPTPRQTRLLESHAGGARFAYNLMLAHVRRQISLGEKPDWTLYAMRR
WWNEWKDEIAPWWRENSKEAYGSAFEWLSQALRNWSDSRKGRRAGRRVGWPKYKS
KRSSVPRFAYTTGSFGLIEDDPKALRLPRIGRVHCMENATERVHGRRIVRMTVSRHAGF
WYAALTVERPTESVPAKNRKRKNHDRQVGVDLGVRTLATLSDGTTFPNPRNYVRTQR
KLRHAQQSLSRRDRGMSHGCGSKRYNRALERVRRIHARIAAQRADNIGKLTTWLADNY
SDISIEDLNVQGMSHNRRLAKHILDADFHEFRRQLEYKTARAGTRLHVIDRWYPSSKTC
SNCGTVKAKLSLSERVYHCEECGLVIDRDVNAAINIQVAGSAPETLNARGGSVGQTRLE
CGTMRHPAKREPSGGDSRVRLGAGLGNEAMQMTSL
ISC1926 (IS607 family) TnpB protein. Length: 412; From NCBI Accession No. AY671948
IS finder database entry: https://isfinder.biotoul.fr/scripts/ficheIS.php?name=ISC1926
(SEQ ID NO: 15)
MERTIKLRVRVDYITYSALKEVEGEYREVLEDAINYGLSNKTTSFTRIKAGVYKTEREKH
KDLPSHYIYTACEDASERLDSFEKLKKRGRSYTEKPSVRKVTVHLDDHLWKFSLDKISIS
TMQGRVFISPTFPKIFWRYYNTEWRIASEARFKLLKGNVVEFFIVFKRDEPKPYEPKGFIP
VDLNEDSVSVLVDGKPMLLETNTKRITLGYEYRRKAITTRRSAEDREVKRKLKRLRERD
KKVVIRRKLAKLIVKEAFESMSAIVLEALPRRPPEHMIKDVKDSQLRLRIYRSAFSSMKN
AIIEKAKEFRVPVVLVNPSYTSSTCPIHGAKIVYQPDGGDAPRVGVCEKGKEKWHRDVV
ALYNLRKRAGDVSPVPLGSKESHDPPTVKLGRWLRAKSLHSIMNEHKMIEMKV

The protein comprising the TnpB protein may additionally comprise one or more effector molecules, and in particular may comprise one or more effector molecules covalently linked to the TnpB protein to form a fusion protein. Fusion proteins according to the disclosure are discussed further below.

The present disclosure also relates to DNA and RNA encoding a protein comprising, consisting essentially of, or consisting of the TnpB protein described herein, from which the protein may be produced by expression. Expression of the DNA or RNA can occur in vitro, ex vivo or in vivo.

### Inactive TnpB Proteins

The protein to be used in the effector complex may also comprise, consist essentially of, or consist of a mutant TnpB protein which has its nuclease activity inactivated, either in part or in full. Such proteins have one or more mutations in the RuvC-like domain of the protein that affect the nuclease activity of the TnpB. In particular, point mutations in RuvC-like domains that remove nuclease activity are already known in the art and have been used to generate mutant Cas12 (Cpf1). The mutations D917A and E1006A of FnCpf1 were reported to completely inactivate the cleavage activity of FnCpf1, while the mutation D1225A significantly reduced nucleolytic activity (Zetsche et al., 2015). Mutations of similar key residues in the RuvC-like domain of the TnpB protein can also be used to remove the nuclease function of the TnpB and to create the inactivated/mutant TnpB proteins described herein. As noted above, and shown in FIG. 12, the RuvC-like domain of TnpB proteins typically contains a conserved D - - - E - - - D motif, which can be mutated. The locations of these residues in each of SEQ ID Nos: 1 to 15 is shown in FIG. 12. For example, within SEQ ID NO: 1 (the TnpB protein from ISDra2) these are D191, E278 and D361. Equivalent residues in other TnpB proteins can be identified using sequence alignment tools, e.g., the Clustal Omega sequence alignment program (https://www.ebi.ac.uk/Tools/msa/clustalo/) (Madeira et al., 2019).

Accordingly, in one example, the inactive mutant TnpB protein may comprise a TnpB protein as described herein, with a mutation of an amino acid residue in the RuvC-like domain such that the nuclease activity is inactivated or partially inactivated. In particular, the mutation may be in one, two or three of the amino acid residues in the conserved D - - - E - - - D motif.

In particular examples, the mutant TnpB protein has a sequence having at least 85%, at least 90%, at least 95%, at least 98%, or at least 99% sequence identity with SEQ ID NO: 1, wherein the sequence is mutated at least at one of positions D191, E278 and D361 of SEQ ID NO: 1 such that the RuvC-like domain is inactivated or partially inactivated.

MIRNKAFVVRLYPNAAQTELINRTLGSARFVYNHFLARRIAAYKESGKG
LTYGQTSSELTLLKQAEETSWLSEVDKFALQNSLKNLETAYKNFFRTVK
QSGKKVGFPRFRKKRTGESYRTQFTNNNIQIGEGRLKLPKLGWVKTKGQ
QDIQGKILNVTVRRIHEGHYEASVLCEVEIPYLPAAPKFAAGVDVGIKD
FAIVTDGVRFKHEQNPKYYRSTLKRLRKAQQTLSRRKKGSARYGKAKTK
LARIHKRIVNKRQDFLHKLTTSLVREYEIIGTEHLKPDNMRKNRRLALS
ISDAGWGEFIRQLEYKAAWYGRLVSKVSPYFPSSQLCHDCGFKNPEVKN
LAVRTWTCPNCGETHDRDENAALNIRREALVAAGISDTLNAHGGYVRPA
SAGNGLRSENHATLVV (SEQ ID NO: 1 with positions
D191, E278 and D361 shown in bold)

In other examples, the mutant TnpB protein has a sequence having at least 85%, at least 90%, at least 95%, at least 98%, or at least 99% sequence identity with one of SEQ ID NOs: 2 to 15, wherein the sequence is mutated at least at one of the boxed amino acid residues shown in FIG. 12 such that the RuvC-like domain is inactivated or partially inactivated.

Effector complexes comprising inactive TnpB proteins may be used simply to block a particular target region comprising a target site in a polynucleotide, e.g., to disturb transcription in the region. They may also be used to detect the presence of a polynucleotide comprising a target sequence in a sample, e.g., in methods where binding of the effector complex to the target site causes a measurable change in a physical or chemical property of a detection system (e.g., in the context of a biosensor).

Inactive TnpB proteins may also be used in effector complexes comprising one or more effector molecules. In these aspects the TnpB protein becomes a carrier for the one or more effector molecules (which may also be termed as “one or more cargo molecules”), to deliver the one or more effector molecules to a particular target region in a polynucleotide. In one example, the one or more effector molecules (particularly when they are effector molecules that are protein-based, e.g. enzymes or protein labels like fluorescent proteins) can be “carried” as part of a fusion protein with TnpB (as discussed further below). Alternatively, or in addition, one or more effector molecules may be “carried” as part of the RNA or bound to the RNA as described further below.

The present disclosure also relates to DNA and RNA encoding a protein comprising, consisting essentially of, or consisting of the inactive TnpB protein described herein, from which the protein may be produced by expression. Expression of the DNA or RNA can occur in vitro, ex vivo or in vivo.

### TnpB Fusion Proteins

As noted above, the effector complex may carry one or more effector molecules in the form of a fusion protein with the TnpB protein. In such examples of the present disclosure, the protein of the effector complex comprises the TnpB protein and one or more effector molecules fused to the N or C terminus of the TnpB protein. Depending on the desired function of the fusion protein in the effector complex, the fusion protein may comprise the TnpB protein or the inactive (mutant) TnpB protein identified above that does not comprise an active nuclease domain.

The one or more effector molecules may be one or more nuclear localisation signals (NLS) which assists the transport of the protein into the nucleus of a cell by the nuclear transport. In particular, such NLS may be used when the target polynucleotide is in the nucleus of a cell. Typically, NLSs are short sequences of positively charged lysines or arginines that are present at or near the N or C terminals of the protein such that when the protein is complexed with the RNA they are exposed on the protein surface. Non-limiting examples of NLSs include the sequence PKKKRKV (SEQ ID No: 18) from the SV40 Large T-antigen, and the bipartite NLS of nucleoplasmin which includes two clusters of basic amino acids KR and four K residues, separated by a spacer of about 10 amino acids (for example KRPAATKKAGQAKKK—SEQ ID NO: 19). Other NLSs are known in the art.

Depending on how the effector complex is to be delivered to the cell, the fusion protein may also comprise cell penetrating peptide—a short peptide that facilitates take up of the fusion protein into a cell.

In addition, or alternatively, the fusion protein may comprise one or more effector molecules. The one or more effector molecules may be: one or more effector molecules capable of modifying the polynucleotide in the target region; one or more effector molecules that are one or more trans-acting factors that are capable of increasing or decreasing transcription of the target region; and/or one or more effector molecules that are capable of labelling the target region.

Methods are already known in the art that utilise Cas9 and Cas12 fusion proteins to deliver one or more effector molecules to a target region (e.g., as described in Knott et al., 2018, and Anzalone et al., 2020). Similar components may be fused to the TnpB protein or the inactive TnpB protein. In particular, the small size of TnpB makes it a good scaffold for the generation of fusion proteins.

In particular, the one or more effector molecules can be selected from an endonuclease, a ribonuclease, a nickase, a base editor, an epigenetic modifier, a transposase, a recombinase, and a reverse transcriptase. In particular, where the base editor is a deaminase, it can be a cytidine deaminase and/or an adenine deaminase. Fusion proteins comprising a cytidine deaminase may also comprise a uracil glycosylase inhibitor.

One or more effector molecules for labelling of the target region may be utilised in the fusion protein. The label may be a reporter enzyme or a fluorescent protein, such as GFP, that can be used to detect the effector complex once the guide RNA has hybridised to the target sequence.

One or more effector molecules for increasing or decreasing transcription or translation of the target region may be utilised in the fusion protein. These may be one or more transcription activators or one or more transcription repressors.

The present disclosure also relates to DNA and RNA encoding a fusion protein comprising the TnpB protein (or the inactive TnpB protein) and the one or more effector molecules described herein, from which the fusion protein may be produced by expression. Expression of the DNA or RNA can occur in vitro, ex vivo or in vivo.

### RNA for Use in the Effector Complex

The present disclosure also relates to an RNA that is capable of binding to the TnpB protein to form the effector complex, and which can guide or direct the effector complex to a target region in a polynucleotide.

In particular the present disclosure provides an RNA comprising:
(i) a protein-binding segment that allows the RNA to bind to a TnpB protein to form an effector complex, and
(ii) a polynucleotide-targeting segment comprising a guide sequence capable of hybridising to a target sequence in a target region of a polynucleotide.

The protein-binding segment of the RNA interacts with the TnpB protein, binding the RNA to the TnpB protein and forming the effector complex. The protein-binding segment may comprise a sequence capable of forming an RNA secondary structure. The protein-binding segment may comprise at least one inverted repeat sequence—a sequence section that is followed downstream by its reverse complement, such that two sections are able to hybridise to form a double-stranded RNA (dsRNA) duplex, such as a hairpin, an imperfect hairpin, or other secondary RNA structure. In particular, the one or more inverted repeat sequence(s) may be one or more at least partially palindromic sequence(s) such that the sequence(s) is/are capable of forming at least one hairpin or at least one imperfect hairpin (which can also be referred to as a stem loops or hairpin loops).

The protein-binding segment can comprise a sequence from a right end (RE) of an insertion sequence in the IS200/IS605 or the IS607 family (in which the thymine residues in the RE DNA sequence are replaced by uracil residues). The RE sequence may be an imperfect palindromic sequence from a mobile genetic element in the IS200/IS605 family. The RE sequence may incorporate part of the terminal sequence of the tnpB gene. The RE sequence may be from the same mobile genetic element as the tnpB from which the TnpB protein in the effector complex is derived. The RE sequence of a particular insertion sequence may be known in the art (e.g., may be available in the ISfinder database, such as those from the same insertion sequences as the TnpB proteins having SEQ ID Nos: 1 to 15 referenced above). Alternatively, the RE sequences may be determined based on sequencing the right end of the insertion sequence that moves with the tnpB gene during transposition. The section of the RE sequence that can be used in the protein-binding segment can be determined in an assay in which the tnpB gene is co-expressed in a suitable host cell (such as E. coli) with the full insertion sequence (optionally with an inactivated tnpA gene where this is present in the insertion sequence), followed by characterisation of the TnpB bound RNA, e.g., by small RNA sequencing, as described in Example 1 herein.

In one example of the protein-binding segment comprises or consists of SEQ ID NO: 16—GAAUCACGCGACUUUAGUCGUGUGAGGUUCAA (which is capable of forming the imperfect hairpin shown in FIG. 1D). This sequence is from the RE of the insertion sequence ISDra2. Accordingly, preferably where the protein described above comprises the TnpB protein with the amino acid sequence SEQ ID NO: 1 (which from the tnpB gene of ISDra2) the protein-binding segment of the RNA comprises or consists of SEQ ID NO: 16.

The polynucleotide-targeting segment of the RNA comprises a guide sequence that is capable of hybridising to, i.e., is complementary to, a target sequence in a target region of a polynucleotide. This segment of the RNA acts to direct or target the effector complex to the target region in the polynucleotide.

The target sequence to which the RNA hybridises may be in single-stranded DNA or may be part of a double-stranded DNA polynucleotide. In examples where the effector complex comprises a mutant/inactive TnpB and is being used to block the target region or to deliver one or more effector proteins to the target region, the target sequence to which the DNA hybridises may be RNA.

(As described herein, where the polynucleotide comprising the target sequence is double-stranded DNA the location at which the site-specific cleavage of the polynucleotide occurs is determined both by the complementary base-pairing between the guide sequence and the target sequence, and by the short TnpB-associated sequence motif (TAM), which interacts with the TnpB protein.)

The guide sequence of the RNA may be between 10 and 30 nucleotides in length, or between 15 and 25 nucleotides in length, and has sufficient complementarity to the target sequence to enable hybridisation between the guide sequence and the target sequence under the particular conditions in which the effector complex is being used. In most situations a high degree of complementarity, of 80% or more is preferred.

The two segments of the RNA are covalently linked as a single RNA molecule, and optionally there may be intervening linker ribonucleotides separating the two segments. The RNA may be arranged 5′ protein-binding segment-(optional linker)-polynucleotide-targeting segment-3′ or 5′ polynucleotide-targeting segment-(optional linker sequence)-protein-binding segment-3′. Preferably the arrangement is 5′ protein-binding segment-(optional linker)-polynucleotide-targeting segment-3′.

Overall, the RNA may be between 50 and 300 nucleotides in length, between 100 and 200 nucleotides in length, or between 140 and 150 nucleotides in length.

It is noted that the RNA is an engineered RNA that is not naturally occurring, i.e., the RNAs are artificially created—the polynucleotide-targeting segment and the protein-binding segment do not occur together in nature.

In particular, in preferred embodiments the guide RNA is complementary to non-bacterial, non-archaeal gene sequences.

The RNA provided by the present disclosure may include chemical modifications, for example to reduce degradation of the RNA in target cells. Techniques for testing modifications in crRNA and tracrRNA used in CRISPR Cas9 and Cas12 systems are already described in the art and can be applied. (For example, Mir et al., 2018.)

The RNA molecule may further comprise segments that enable the RNA to bind to one or more effector molecules that are to be delivered to the target region comprising the target sequence of the polynucleotide. Aptamers such as MS2 hairpins or PP7 hairpins can be engineered into the RNA, to which an effector molecule (e.g., MS2 RNA coat protein MCP fused to a fluorescent protein) can be tethered or bound, e.g., in a manner that has been described in the art for dCas9 (Sajwan S, et al., 2019; Ma H, et al., 2018; Ma et al., 2016).

The present disclosure also relates to DNA encoding the RNA described herein, from which the RNA may be produced by expression. Expression of the DNA can occur in vitro, ex vivo or in vivo.

### The Effector Complex

Also provided by the present disclosure are effector complexes which comprise the protein and the RNA identified above. These are guided by the RNA to a target sequence in a target region of a polynucleotide—the RNA comprising a polynucleotide-binding segment comprising a guide sequence that hybridises to the target sequence of the polynucleotide.

The polynucleotide to which the effector complex is directed may be double-stranded DNA, or single-stranded DNA. Preferably the polynucleotide is double-stranded DNA. In examples where the effector complex comprises a mutant/inactive TnpB and is being used to block the target region or to deliver one or more effector proteins to the target region, the target sequence to which the effector complex is directed may be RNA.

Where the effector complex comprises a TnpB with an active nuclease site, the effector complex is able to cleave the DNA in the target region. The cleavage may be within 30 bp from the end of the target site. The cleavage site may be 5′ of the target sequence on the strand comprising the target sequence.

In one example the effector complex is able to cleave the double-stranded polynucleotide generating a staggered double-stranded break. The 5′ overhang may, for example, be 4 or 5 nucleotides in length. Alternatively, the effector complex may cleave the double-stranded polynucleotide to generate blunt ends.

The effector complex of the present disclosure may be an engineered, non-naturally occurring complex. In particular, the RNA and the protein of the complex do not occur together in nature.

The effector complex may be in an isolated or purified form.

In one example of the present disclosure the effector complex is bound to a solid support. In particular, the effector complex can be bound to a solid support in a biosensor that can be used to detect the presence of a target sequence (e.g., as has been shown for Cas9-based effector complexes immobilise on a graphene field-effector transistor in Hajian et al., (2019)). Suitable methods for conjugating proteins to a solid surface, which may be utilised to conjugate the effector complex to a solid surface, are known in the art. In one example the effector complex can comprise a fusion protein as described above, comprising a TnpB protein (or inactivated TnpB protein) and a peptide tag that can be utilised to capture the effector complex on the surface of a solid support.

The effector complex of the present disclosure may be produced in vitro, ex vivo or in vivo. In particular, the method can comprise assembly of the effector complex from the RNA and the protein described herein in cells or in vitro in a cell-free system.

Where the effector complex is produced in cells, the method may comprise providing the following in the cell:
(i) the RNA described herein, and DNA encoding the protein described herein;
(ii) the protein described herein, and DNA encoding the RNA described herein;
(iii) DNA encoding the protein described herein and DNA encoding the RNA described herein;
(iv) RNA (mRNA) encoding the protein described herein and the RNA described herein; or
(v) RNA (mRNA) encoding the protein described herein and DNA encoding the RNA described herein.

Where the effector complex is produced in vitro in a cell-free system, the method may comprise in vitro expression of DNA encoding the RNA, in vitro expression of DNA encoding the protein, or in vitro expression of both DNA encoding the RNA and DNA encoding the protein.

The DNA encoding the protein and/or the DNA encoding the RNA may comprise one or more regulatory elements for regulating expression of the DNA in the cell or in the cell-free system. In particular, the DNA encoding the protein may comprise at least one first regulatory element operably linked to the DNA sequence encoding the protein and/or the DNA encoding the RNA may comprise at least one second regulatory element operably linked to the DNA sequence encoding the RNA. By “operably linked” it is meant that the regulatory elements are positioned in the DNA sequence so as to be able to be able to affect expression of the DNA sequences encoding the RNA and the protein. The regulatory elements may be promoters, enhancers, internal ribosome entry sites and other expression control elements. These can be selected depending on the cell type being used to express the RNA and the protein, or the other components selected for use in the in vitro cell-free system.

The DNA sequences disclosed herein may be incorporated in a vector. In particular, the vector may be used for expressing, maintaining and/or propagating the DNA sequences. Suitable vectors include plasmids and viral vectors. The viral vectors may be selected from a retrovirus vector, a lentivirus vector, an adenovirus vector, an adeno-associated virus (AAV) vector or a herpes simplex virus vector. In particular, viral vectors already known in the art for use in combination with the CRISPR-Cas9 and CRISPR-Cas12 systems can be used (as described for example in Xu et al., 2019). In a preferred example the viral vector is an AAV viral vector. In particular, due to the relatively small size of the TnpB protein, the AAV viral vector can particularly be utilized where the TnpB (or inactivated TnpB) for the effector complex is part of a fusion protein carrying one or more effector molecules.

The present disclosure also provides host cells transfected with the DNA encoding the RNA and/or the DNA encoding the protein described herein. The host cells can be used for in vitro expression of the DNA encoding the RNA and/or the DNA encoding the protein described herein, and in particular for use in the production of the effector complex. The host cell comprises the DNA encoding the RNA and/or the DNA encoding the protein described herein. The DNA may be integrated into the genome of the host cell so as to be replicated along with the host genome. Alternatively, the DNA may remain on a vector that has been used to transfect the cell.

The DNA can be defined as being foreign to the host cell, i.e., a host cell comprising the DNA does not occur in nature.

In some examples the host cell is an isolated cell.

In some examples the host cell is not a totipotent human embryonic stem cell.

In some examples the host cell is not a human oocyte.

In some examples, the host cell does not contain a target sequence complementary to the guide sequence of the RNA.

The host cell may be a cell from a cell line.

In one aspect of the disclosure the host cell can be utilised to produce the effector complex described here so that the effector complex can then be used in the methods discussed below.

In an alternative aspect of the disclosure the production of the effector complex can occur as part of the methods and uses of the effector complex discussed herein.

Both of these aspects may involve the following system, which is also provided by the present disclosure:

A system for modifying a target region in a polynucleotide, wherein the target region comprises a target sequence, the system comprising:
a) a protein comprising or consisting of a TnpB protein, or DNA or RNA encoding said protein, and
b) an RNA, or DNA encoding the RNA, the RNA comprising:
   (i) a polynucleotide-targeting segment comprising a sequence that is complementary to the target sequence; and
   (ii) a protein-binding segment that binds the TnpB protein.

The system may comprise (a) the protein and (b) the RNA; (a) the DNA encoding the protein, and (b) the DNA encoding the RNA; (a) DNA encoding the protein and (b) the RNA; (a) the protein and (b) DNA encoding the RNA; (a) RNA (mRNA) encoding the protein and (b) the RNA; or (a) RNA (mRNA) encoding the protein and (b) DNA encoding the RNA. In particular examples, (a) and (b) are both RNA (for example as has been shown for Cas9 (Gillmore et al., 2021)).

The RNA and protein comprising the TnpB are as described herein. In particular, the protein can be the fusion protein described herein. The TnpB can be the inactivated TnpB described herein.

In the system, (a) and/or (b) can be comprised in at least one vector. In one example (a) and (b) are comprised in the same vector. In an alternative example (a) and (b) are comprised in separate vectors.

The vectors may be non-viral vectors of viral vectors. In particular, the non-viral vector may be at least one plasmid, and/or at least one non-viral particle such as a liposome or an exosome.

Alternatively, the at least one viral vector may be selected from a retrovirus vector, a lentivirus vector, an adenovirus vector, an adeno-associated virus (AAV) vector or a herpes simplex virus vector. As noted above, in particular where the protein is a fusion protein comprising the TnpB and one or more effector molecules, an AAV vector may be preferred.

The system of the present disclosure is an engineered, non-naturally occurring system.

The system may be in the form of a kit with (a) and (b) separately packaged, optionally the kit being packaged with instructions for use.

The system and effector complexes described above can be comprised in the vectors described above for delivery to cells in vitro, ex vivo or in vivo.

In addition, the system or the effector complex either alone or as part of a vector can be delivered by microinjection or via electroporation. In particular, the vector may be a liposome.

The system or the effector complex may be delivered chemically, via lipofection (lipid-mediated), transfection (cationic polymer mediated) or by calcium phosphate transfection.

Viral vectors may also be utilised for delivery, including lentiviral vectors, retroviral vectors and AAV vectors.

In particular, delivery systems based on those already described for the CRISPR Cas9 and Cas12 systems can be utilised (see e.g., https://blog.addgene.org/crispr-101-mammalian-expression-systems-and-delivery-methods).

As noted above, the fusion protein used in the effector complex may comprise a cell penetrating peptide, to facilitate uptake of the effector complex, or the fusion protein, by the cells.

### Methods and Uses

The effector complexes and or systems described herein may be used in methods for cleaving, modifying, labelling or controlling expression from a target region in a polynucleotide, where the target region comprising a target sequence.

In particular, the method may be method for delivering an effector complex to a target region in a polynucleotide, wherein the target region comprises a target sequence, the effector complex comprising:
(a) a protein comprising or consisting of a TnpB protein; and
(b) an RNA comprising:
   (i) a polynucleotide-targeting segment comprising a guide sequence capable of hybridising to the target sequence; and
   (ii) a protein-binding segment that allows the RNA to bind to the TnpB protein,
      the method comprises contacting the effector complex with the polynucleotide and allowing the guide sequence to hybridise to the target sequence so as to deliver the effector complex to the target region. The effector complex may comprise one or more effector molecules as described herein, which are delivered to the target region.

In a further aspect the method may be a method for cleaving a polynucleotide with an effector complex, wherein the polynucleotide comprises a target sequence, the effector complex comprising:
(a) a protein comprising or consisting of a TnpB protein; and
(b) an RNA comprising:
   (i) a polynucleotide-targeting segment comprising a guide sequence capable of hybridising to the target sequence; and
   (ii) a protein-binding segment that allows the RNA to bind the TnpB protein to form the effector complex,
      wherein the method comprises contacting the polynucleotide with the effector complex and allowing the TnpB protein to cleave the polynucleotide.

Where the polynucleotide is double-stranded DNA, the cleavage may produce a staggered double-stranded break with a 5′ overhang. Alternatively, the cleavage may produce a blunt-ended double stranded break.

The contacting step of the method may occur in a cell under conditions that allow for non-homologous end joining (NHEJ) or homology-directed repair (HDR) of the cleaved polynucleotide so as to edit the sequence of the polynucleotide. Further, the method may further comprise contacting the polynucleotide with a donor polypeptide for HDR. Suitable methods for achieving NHEJ and HDR that are known in the art for Cas9 and Cas12 systems are also suitable in the present case (e.g., see Maresca et al., 2013).

In the methods according to these aspects the polynucleotide may be a double stranded DNA and may comprise a TnpB-associated sequence motif 5′ of the target sequence (as described above) with which the TnpB interacts.

Alternatively, the polynucleotide may be single-stranded DNA.

In one example of the methods of the disclosure, the polynucleotide may be within a cell. The cell may be a prokaryotic cell or a eukaryotic cell. Where the cell is a eukaryotic cell, it may be non-human animal cell, a human cell or a plant cell. In particular the cell may be a stem cell, such as an induced pluripotent stem cell.

Like the Cas9 and Cas12 systems, the methods of the present disclosure have particular utility in plant cells. In particular, the present disclosure includes a method for producing a plant comprising cells with a modified polynucleotide, the method comprising contacting a plant cell with the system described herein or the effector complex described herein, thereby modifying a target region of said polynucleotide, and regenerating a plant from said plant cell, wherein the modified target region is in a gene of interest in said cell, and wherein the modification is associated with a trait of interest.

The effector complex, the system and the DNA encoding the components of the complex and the system may be for use as a medicament in an individual. Alternatively, they may be used for a method of diagnosis in an individual.

Alternatively, the effector complex, the system and the DNA encoding the components of the complex and the system may be used in in vitro or ex vivo methods to determine the presence of a polynucleotide comprising a target sequence in a sample, or to modify a target region of a polynucleotide.

The disclosure will now be described in more detail, by way of example only, with reference to the following experimental work.

### EXAMPLES

### Materials and Methods

### Engineering TnpB Expression Vectors

pTWIST-ISDra2 plasmid containing the IS200/IS605 ISDra2 system of Deinococcus radiodurans R1 (GenBank AE000513.1) cloned as a synthetic DNA fragment under T7 promoter was obtained from Twist Biosciences. To obtain pGD3 plasmid containing ISDra2 variant with a deletion within tnpA gene. pTWIST-ISDra2 plasmid was pre-cleaved with NdeI (Thermo Fisher Scientific). 5′-overhangs filled-in using T4 DNA Polymerase (Thermo Fisher Scientific) and self-circularized with T4 DNA Ligase (Thermo Fisher Scientific). For TnpB purification two pBAD-derived expression vectors were constructed using NEBuilder HiFi DNA Assembly kit (New England Biolabs): pTK120-ISDra2-TnpB contained tnpB encoding sequence fused to N-term 10×His-TwinStrep-MBP protein purifications tag while pTK151 contained tnpB fused to N-term 6×His-MBP and C-term StrepTag II encoding sequences. To obtain reRNA expression vector (pGB71) used for TnpB complex purification, reRNA encoding sequence carrying T7 promoter at the 5′-end and HDV (hepatitis delta virus) ribozyme and T7 terminator at the 3′-end (assembled by PCR from synthetic oligonucleotides) was cloned into pACYC184 vector over HindIII and BclI restriction sites (Thermo Fisher Scientific). pGB74-78 plasmids used for TnpB complex expression in 7N plasmid library cleavage and plasmid interference assays, contained reRNA and tnpB encoding sequences under T7 and T7lac promoters, respectively. pGB74-78 plasmids were obtained by cloning reRNA encoding fragment over Bsu15I and EcoRI (Thermo Fisher Scientific) sites and tnpB over NdeI and XhoI (Thermo Fisher Scientific) sites into the pET-Duet1 vector (Novagen). For genome editing experiments in human HEK293T cells. plasmid vectors pRZ122-127. the derivatives of pX458 plasmid (gift from Feng Zhang. Addgene plasmid #48138). encoding reRNA (targeting 20 bp sites in human genomic DNA) and tnpB (fused at 3′-end with SV40 NLS-T2A-GFP) under U6 and CAG promoters, respectively. were constructed using NEBuilder HiFi DNA Assembly kit (New England Biolabs). Phusion Site-Directed Mutagenesis Kit (Thermo Fisher Scientific) was used to obtain plasmid variants with mutated RuvC active site.

### Expression and Purification of TnpB RNP Complex

For initial TnpB protein expression and pre-purification, E. coli BL21-AI cells were transformed with pTK120-ISDra2-TnpB alone or co-transformed with pGD3 (encoding ISDra2 transposon with deletion within tnpA gene) and grown at 37° C. in LB broth supplemented with ampicillin (100 μg/ml) or ampicillin (100 μg/ml) and chloramphenicol (50 μg/ml), respectively. After culturing to an OD₆₀₀ of 0.6-0.8 protein expression was induced with 0.2% arabinose and the cells were grown for additional 16 h at 16° C. temperature. Next day, the cells were pelleted by centrifugation, resuspended in 20 mM Tris-HCl, pH 8.0 at 25° C., 250 mM NaCl, 5 mM 2-mercaptoethanol, 25 mM imidazole, 2 mM PMSF and 5% (v/v) glycerol containing buffer and disrupted by sonication. After removing cell debris by centrifugation, the supernatant was loaded onto the Ni²⁺-charged HiTrap chelating HP column (GE Healthcare) and proteins were eluted with a linear gradient of increasing imidazole concentration from 25 mM to 500 mM in 20 mM Tris-HCl, pH 8.0 at 25° C., 500 mM NaCl, 5 mM 2-mercaptoethanol and 5% (v/v) glycerol buffer. The fractions containing TnpB were pooled, dialyzed against 20 mM Tris-HCl, pH 8.0 at 25° C., 250 mM NaCl, 2 mM DTT and 50% (v/v) glycerol and stored at −20° C. The obtained pre-purified TnpB samples were used for nucleic acid extraction and analysis.

For increased expression and yield of TnpB RNP complex, E. coli BL21-AI cells were transformed with reRNA (pGB71) and TnpB (pTK151) or TnpB^{D191A} (pTK152) expression vectors and grown in LB broth supplemented with ampicillin (100 μg/ml) and chloramphenicol (50 μg/ml) at 37° C. After culturing to an OD₆₀₀ of 0.6-0.8 protein expression was induced with 0.2% arabinose and cells were grown for additional 16 h at 16° C. Next day, the cells were pelleted by centrifugation, resuspended in 20 mM Tris-HCl, pH 8.0 at 25° C., 500 mM NaCl, 5 mM 2-mercaptoethanol, 25 mM imidazole, 2 mM PMSF and 5% (v/v) glycerol containing buffer and disrupted by sonication. After removing cell debris by centrifugation, the supernatant was loaded onto the Ni²⁺-charged HiTrap chelating HP column (GE Healthcare) and bound proteins were eluted with a linear gradient of increasing imidazole concentration from 25 to 500 mM in 20 mM Tris-HCl, pH 8.0 at 25° C., 500 mM NaCl, 5 mM 2-mercaptoethanol and 5% (v/v) glycerol buffer. The fractions containing TnpB RNP complexes were pooled and the 6×His-MBP tag was cleaved by overnight incubation with TEV protease at 8° C. Next, the reaction mixture was loaded onto the StrepTrap column (GE Healthcare), washed with 20 mM Tris-HCl, pH 8.0 at 25° C., 150 mM NaCl, 5 mM 2-mercaptoethanol and 5% (v/v) glycerol buffer and bound TnpB complex eluted with 2.5 mM d-desthiobiotin solution. Fractions containing TnpB were pooled, loaded on HiTrap heparin HP column (GE Healthcare) and eluted using a linear gradient of increasing NaCl concentration from 0.15 M to 1.0 M. Obtained TnpB complex fractions were pooled, concentrated up to 0.5 ml using

Amicon Ultra-15 centrifugal filter unit (Merck Millipore) and loaded on Superdex 200 10/300 GL (GE Healthcare) gel filtration column equilibrated with 20 mM Tris-HCl, pH 8.0 at 25° C., 250 mM NaCl, 5 mM 2-mercaptoethanol buffer. Peak fractions containing TnpB RNP complexes were pooled and dialyzed against 20 mM Tris-HCl, pH 8.0 at 25° C., 250 mM NaCl, 2 mM DTT and 50% (v/v) glycerol containing buffer and stored at −20° C. The concentration of the TnpB RNP complex was determined by quantifying intensity of protein bands in SDS-PAGE gels and comparing them to protein standard of known concentration.

### Molecular Mass Measurements by Mass Photometry

Measurement coverslips (No. 1.5 H, 24×50 mm, Marienfeld) were cleaned by sequential sonication for 5 min in MilliQ water, isopropanol and MilliQ water and then dried using a clean stream of nitrogen gas. Cleaned coverslip was mounted onto the OneMP mass photometer (Refeyn Ltd.) and a CultureWell™ Reusable Gasket (Grace Bio-Labs) was placed on top. A gasket well was filled with 10 μl of 20 mM Tris-HCl, pH 8.0 at 25° C. and 250 mM NaCl buffer, 10 μl of the diluted TnpB RNP complex sample (˜60 nM) was added and the adsorption of biomolecules was monitored for 120 s using the AcquireMP software (Refeyn Ltd). For converting the measured ratiometric contrast into molecular mass, Un1Cas12f1 protein (Karvelis et al., 2020) and its oligomers ranging from 60 to 250 kDa (monomer to tetramer) were used for calibration. Samples were measured in triplicates. Mass photometry movies were analyzed using the DiscoverMP (Refeyn Ltd).

### TnpB-Bound Nucleic Acids Extraction and Analysis

To extract TnpB bound nucleic acids, first, 100 μl of pre-purified TnpB samples were incubated with 5 μl (20 mg/ml) of Proteinase K (Thermo Fisher Scientific) for 45 min at 37° C. in 1 ml of 10 mM Tris-HCl, pH 7.5 at 37° C., 5 mM MgCl₂, 100 mM NaCl, 1 mM DTT and 1 mM EDTA reaction buffer. Next, the nucleic acids were extracted by phenol:chloroform:isoamyl alcohol (25:24:1) solution and the aqueous phase was additionally treated with chloroform to remove any remaining phenol. The solution containing nucleic acids was split into fresh tubes (198 μl each), then 2 ul of RNase I (10 U/μl) (Thermo Fisher Scientific) or DNase I (10 U/μl) (Thermo Fisher Scientific) were added, and reactions were incubated for 45 min at 37° C. Reaction products were mixed with 2×RNA Loading Dye (Thermo Fisher Scientific), separated on TBE-Urea (8 M) 15% denaturing polyacrylamide gel using 0.5×TBE electrophoresis buffer (Thermo Fisher Scientific) and visualized with SYBR™ Gold (Thermo Fisher Scientific).

### RNA Isolation From TnpB RNP Complex

For TnpB bound RNAs extraction, 100 μl of pre-purified TnpB complex was incubated with 5 μl (20 mg/ml) of Proteinase K (Thermo Fisher Scientific) for 45 min at 37° C. in 1 ml of a reaction buffer containing 10 mM Tris-HCl, pH 7.5 at 37° C., 5 mM MgCl₂, 100 mM NaCl, 1 mM DTT and 1 mM EDTA. The DNA was digested by adding 10 μl of DNase I (10 U/μl) (Thermo Fisher Scientific) followed by an additional 45 min incubation at 37° C. and subsequent purification using GeneJET RNA Cleanup and Concentration Micro Kit (Thermo Fisher Scientific). Next, 3 μg of purified RNA was phosphorylated using 1 μl (10 U/μl) of PNK (Thermo Fisher Scientific) in 1× Reaction Buffer A (Thermo Fisher Scientific), supplemented with 1 mM ATP at 37° C. for 30 min in 20 ul reaction volume, and purified with a GeneJET RNA Cleanup and Concentration Micro Kit (Thermo Fisher Scientific).

### RNA Sequencing and Analysis

RNA libraries were prepared using Collibri™ Stranded RNA Library Prep Kit for Illumina™ Systems (Thermo Fisher Scientific) according to the manufacturer's instructions for small RNAs (protocol MAN0025359), pooled in an equimolar ratio and pair-end sequenced (2×75 bp) using MiSeq Reagent Kit v2, 300-cycles (Illumina) on a MiSeq System (Illumina). The pair-end reads shorter than 20 bp were filtered with Cutadapt (Martin, 2011). The remaining reads were mapped to the transposon encoding plasmid (pTWIST-ISDra2) using BWA (Li and Durbin, 2009) and converted to the .bam file format with SAMtools (Li et al., 2009). The resulting coverage data was visualized using IGV (Robinson et al., 2011).

### Detecting TnpB dsDNA Cleavage and TAM Recognition

PAM determination assay developed previously for Cas9 and Cas12 effectors (Karvelis et al., 2015, 2019, 2020) was adopted for establishment of TnpB dsDNA cleavage requirements and TAM sequence. Briefly, tnpB gene and reRNA constructs, targeting 16 bp or 20 bp sequences in plasmid library, adjacent to a 7N randomized region, were cloned into a pET-duct1 (MilliporeSigma) vector (pGB77-78). Next, E. coli ArcticExpress (DE3) cells were transformed with TnpB RNP encoding plasmids and the cells were grown in LB broth supplemented with ampicillin (100 μg/ml) and gentamicin (10 μg/ml). After reaching OD₆₀₀ of 0.5, TnpB expression was induced with 0.5 mM IPTG and the culture was incubated overnight at 16° C. The cells from 10 ml of overnight culture were collected by centrifugation, re-suspended in 1 ml of lysis buffer (20 mM phosphate, pH 7.0, 0.5 M NaCl, 5% (v/v) glycerol, 2 mM PMSF) and lysed by sonication. Cell debris was removed by centrifugation and 10 μl of the supernatant, containing TnpB RNPs, was used directly for plasmid library digestion. Briefly, lysate was mixed with 1 μg of 7N randomized plasmid library (pTZ57) in 100 μl of reaction buffer (10 mM Tris-HCl, pH 7.5 at 37° C., 100 mM NaCl, 1 mM DTT and 10 mM MgCl₂) and incubated for 1 h at 37° C. Cleaved DNA ends were repaired by adding 1 μl of T4 DNA polymerase (Thermo Fisher Scientific) and 1 μl of 10 mM dNTP mix (Thermo Fisher Scientific), and incubating at 11° C. for 20 min, followed up by heating it up to 75° C. for 10 min. Next, 3′-dA overhangs were added by incubating the reaction mixture with 1 μl of DreamTaq polymerase (Thermo Fisher Scientific) and 1 μl of 10 mM dATP (Thermo Fisher Scientific) for 30 min at 72° C. RNA was removed by adding 1 μl of RNase A (Thermo Fisher Scientific) and incubating the reaction mixture for 15 min at 37° C. with, followed by DNA purification using GeneJet PCR Purification kit (Thermo Fisher Scientific). Next, 100 ng of the purified cleavage products were mixed with 100 ng of dsDNA adapter containing a 3′-dT overhang (100 ng) and incubated for 1 h at 22° C. with 1 μl T4 DNA ligase (Thermo Fisher Scientific) in 20 μl reaction volume. Next, the adapter bearing cleavage products were PCR amplified and gel purified using GeneJet Gel Purification kit (Thermo Fisher Scientific). DNA libraries were prepared using Collibri™ PS DNA Library Prep Kit for Illumina™ Systems (Thermo Fisher Scientific) according to the manufacturer's instructions, pooled in an equimolar ratio and pair-end sequenced (2×150 bp) using MiSeq Reagent Kit v2, 300-cycles (Illumina) on a MiSeq System (Illumina).

Double-stranded DNA cleavage by TnpB RNP complex was evaluated by examining the adapter ligation at the targeted sequence in 7N plasmid library. This was accomplished by extracting and counting all reads containing adapter ligated at the 0-30 bp target positions next to 7N region by identifying 10 bp perfectly matching sequences derived from the adapter and the plasmid backbone. The reads exhibiting elevated frequency of adapter ligation in the target region (20-21 bp from 7N randomized sequence) were used for 7N sequences (TAM) extraction and visualization using WebLogo (Crooks, 2004)). The Python scripts used in cleavage position identifications and TAM characterization are provided at GitHub repository (https://github.com/tkarvelis/Nuclease_manuscript).

### DNA Substrates for In Vitro TnpB Cleavage Reactions

Plasmid DNA substrates (pGB72-73) used in in vitro cleavage assays were obtained by cloning synthetic oligoduplexes (Invitrogen) into pSG4K5 plasmid (gift from Xiao Wang, Addgene plasmid #74492) pre-cleaved with EcoRI and NheI restriction endonucleases (Thermo Fisher Scientific).

Synthetic linear DNA substrates were 5′-end labeled by incubating 1 μM of oligonucleotide (Thermo Fisher Scientific) with 1 μl (10 U/μl) of PNK (Thermo Fisher Scientific) and ³²P-γ-ATP (PerkinElmer) at 37° C. for 30 min in 7.5 μl of 1× Reaction buffer A (Thermo Fisher Scientific). Oligoduplexes (100 nM) were obtained by combining ³²P-labeled and unlabeled complementary oligonucleotides (1:1.5 molar ratio) followed by heating to 95° C. and slow cooling to room temperature.

### DNA Cleavage Assays

Plasmid DNA cleavage reactions were initiated by mixing 100 nM TnpB RNP complex with 3 nM plasmid DNA (pGB72-73) in the reaction buffer containing 10 mM Tris-HCl, pH 7.5 at 37° C., 10 mM MgCl₂, 1 mM DTT, 1 mM EDTA, 100 mM NaCl, followed by 60 min incubation at 37° C. (if not indicated differently). The reactions were quenched by mixing with 3× loading dye solution (0.01% Bromophenol Blue and 75 mM EDTA in 50% (v/v) glycerol) and analyzed by agarose gel electrophoresis and ethidium bromide staining. The linearized plasmid DNA substrate was obtained by cleavage with NdeI endonuclease (Thermo Fisher Scientific).

Cleavage reactions with synthetic oligoduplexes were initiated by combining 100 nM TnpB RNP complex with 1 nM radiolabeled substrate in 100 μl Tris-HCl, pH 7.5 at 37° C., 1 mM EDTA, 1 mM DTT, 10 mM MgCl₂, 100 mM NaCl reaction buffer at 37° C. Aliquots of 10 μl were removed from the reaction mixture at timed intervals (0 min, 1 min, 5 min, 15 min and 60 min), quenched with 1.8× volume of loading dye (95% (v/v) formamide, 0.01% Bromophenol Blue and 25 mM EDTA) and subjected to denaturing gel electrophoresis (20% polyacrylamide containing 8.5 M urea in 0.5×TBE buffer). Gels

### Plasmid Interference Assay

Plasmid interference assays were performed in E. coli Arctic Express (DE3) strain bearing TnpB and reRNA encoding plasmids (pGB74-76). The cells were grown at 37° C. to an OD600 of ˜0.5 and electroporated with 100 ng of target plasmid (pGB72), engineered from pSG4K5 (gift from Xiao Wang, Addgene plasmid #74492). After 1 h, co-transformed cells were further diluted by serial of 10× fold dilutions and grown at 25° C. 30° C. or 37° C. on plates containing IPTG (0.1 mM), gentamicin (10 μg/ml), carbenicillin (100 μg/ml) and kanamycin (50 μg/ml) for 16-44 h.

### TnpB Induced DNA Cleavage in HEK293T Cells

HEK293T cells purchased from ATCC (catalogue number CRL-3216) were cultivated in Dulbecco's Modified Eagle Medium (DMEM) (Gibco) supplemented with 10% foetal bovine serum (Gibco), penicillin (100 U/ml) and streptomycin (100 μg/ml) (Thermo Fisher Scientific). A day prior transfection the cells were plated in a 24-well plate at a density of 1.4×10⁵ cells/well. The transfection mixture was prepared by mixing 1 μg of plasmid encoding NLS-tagged TnpB and its reRNA (pRZ122-127) with 100 μl of serum-free DMEM and 2 μl of TurboFect transfection reagent (Thermo Fisher Scientific). After 15 min incubation at room temperature transfection mixture was added dropwise to the cells. Transfected cells were grown for 72 h at 37° C. and 5% CO₂.

### Indels Characterization

Transfected HEK293T cells were trypsinized and their genomic DNA was extracted using QuickExtract solution (Lucigen). Two rounds of PCR were performed to amplify the DNA region surrounding each target site and add the sequences required for Illumina sequencing and indexing. Briefly, 1-4 μl of DNA lysate was used in a primary PCR with primers specific to the targeted genomic locus that were 5′ tailed with Illumina Read1 and Read2 sequences in a final volume of 20 μl using Hot Start Phusion polymerase (Thermo Fisher Scientific). The thermocycler setting consisted of initial denaturation at 98° C. for 30 s, 15 cycles of 98° C. for 15 s, 56.8° C. for 15 s, 72° C. for 30 s, and final incubation at 72° C. for 5 min. The resulting amplicons were cleaned using 1.8× volume of magnetic beads (Lexogen) and eluted in 30 μl. Six μl of the eluted mixture was used as a template for a second round of PCR in a final volume of 30 μl to index and add P5 and P7 adapters required for Illumina sequencing using Lexogen PCR Add on Kit (Lexogen) with i7 6 nt Index Set (Lexogen). The thermocycler setting consisted of initial denaturation at 98° C. for 30 s, 15 cycles of 98° C. for 10 s, 65° C. for 20 s, 72° C. for 30 s, and final incubation at 72° C. for 1 min. To ensure the purity of the PCR products an additional cleanup with 0.9× volume of magnetic beads (Lexogen) was performed. Barcoded and purified DNA samples were quantified by Qubit 4 Fluorometer (Thermo Fisher Scientific), analyzed using BioAnalyzer (Agilent), pooled in an equimolar ratio and pair-end sequenced (2×75 bp) using MiniSeq High Output Reagent Kit, 150-cycles (Illumina) on a MiniSeq System (Illumina). Insertion or deletion mutations (INDELs) were analyzed using CRISPResso2 (Clement et al., 2019) with the following parameters: minimum of 70% homology for alignment to the amplicon sequence, quantification window of 10 bp, ignoring substitutions to avoid false positives and phred33 score>10 for average read and single base pair quality.

### Example 1—Establishing the Biochemical Function of TnpB in D. Radiodurans ISDra2 Transposable Element

Insertion sequences (ISs) are simple, widespread mobile genetic elements (MGEs) that only contain genes related to transposition and the regulation of transposition. Transposable elements of the IS200/IS605 family are among the simplest and ancient mobile genetic elements (MGE) (Siguier et al., 2014). Typically, they carry subterminal palindromic elements (LE and RE) at MGE ends and mnpA and tnpB genes in different configurations. However, some MGEs of this family contain stand-alone tnpA or tnpB genes (ISfinder database) (Siguier et al., 2006). The best experimentally characterized IS608 and IS200/IS605 MGEs of Helicobacter pylori (Hp) and Deinococcus radiodurans (Dra) ISDra2, respectively, consist of partially overlapping tnpA and tpB genes flanked by left end (LE) and right end (RE) imperfect palindromic sequences (FIG. 1A) (Kersulyte et al., 2002; Pasternak et al., 2010). Transposition is coupled to DNA replication and occurs via “peel and paste” mechanism including an obligatory single-stranded DNA intermediate (Hoang et al., 2010).

The TnpA transposase encoded by tnpA is sufficient to promote IS mobility both in cells and in vitro. The TnpA tyrosine Y1 transposase catalyzes both the excision and insertion of the ssDNA intermediate. TnpA is extremely small (˜18 kDa) protein that forms a dimer and contains a composite active site made of catalytic tyrosine in one monomer and metal binding HUH motif in the other monomer. It cuts transposon encoding DNA strand near “TTAC” (IS608) or “TTGAC” (ISDra2) sequences generating a circular single-stranded (ss) DNA intermediate (FIG. 1B) (Guynet et al., 2008; Pasternak et al., 2010). The integration reaction occurs specifically into ssDNA near the same sequences completing the transposition cycle without target site duplication (Guynet et al., 2008; Pasternak et al., 2010). Interestingly, the target site selection occurs through the base pairing interactions involving transposon LE element sequence rather than by the direct sequence readout by TnpA (Barabas et al., 2008; He et al., 2011). The molecular mechanism of transposition in IS607 family is less well understood: it requires TnpA serine family transposase and may involve double-stranded (ds) DNA intermediate (Boocock and Rice, 2013; Chen et al., 2018; Kersulyte et al., 2000).

Although the TnpA function in transposition is well established, the role of TnpB remains elusive. TnpB is not essential for transposition and is thought to be involved in the negative regulation of transposon excision and insertion (Kersulyte et al., 2000, 2002; Pasternak et al., 2013). Intriguingly, bioinformatic identification of the conserved RuvC-like active site in TnpB sequence, triggered speculations that TnpB can be an ancestor of Cas9 and Cas12 nucleases adopted by CRISPR-Cas systems (Kapitonov et al., 2016; Makarova et al., 2020). However, neither the role of RuvC-motif in transposition nor nuclease activity of TnpB has been experimentally demonstrated.

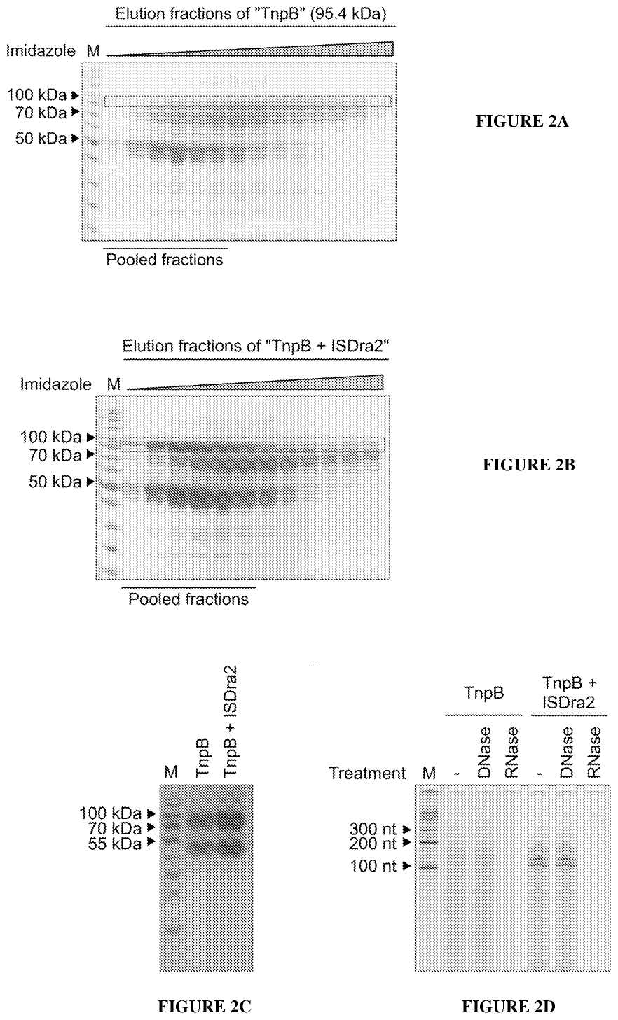

To establish the biochemical function of the TnpB in D. radiodurans ISDra2 transposable element, we aimed to isolate and biochemically characterize the TnpB protein. To this end we expressed in E. coli tnpB gene (1227 bp) fused to the sequence encoding 10×His-MBP (maltose binding protein) purification tag. Initial attempts to purify TnpB from cell extracts by the Ni²⁺-affinity chromatography revealed extremely low yields of intact TnpB protein (FIG. 2A). However, co-expression of tnpB with a full ISDra2 transposon (with inactivated tnpA) resulted in the significant TnpB yield increase suggesting that some transposon elements may contribute to stable TnpB expression (FIGS. 2B and 2C). Subsequent analysis of TnpB samples revealed that RNA co-purified with TnpB (FIG. 2D). To characterize TnpB bound RNAs we performed small RNA sequencing (sRNA-seq) which revealed the enrichment of non-coding RNAs (˜150 nt) derived from ISDra2 transposon RE element that we named reRNAs (FIGS. 1C and 1D). The reRNA co-purified with TpnB matched to the 3′-end of the tnpB gene and RE sequence, except the last ˜16 nt at the 3′-end which derived from the plasmid DNA sequence flanking the IS200/IS605 transposon (FIG. 1D). The enrichment of non-coding RNAs associated with tnpB encoding IS200/IS605 family transposons has been reported previously for Halobacterium salinarum (Gomes-Filho et al., 2015). Taken together, these data show that TnpB forms the ribonucleoprotein (RNP) complex with transposon 3′-end derived reRNA similar to the Cas9 or Cas12 complex with gRNA. In the latter case the variable sequence part of the gRNA corresponds to the spacer sequence in the CRISPR array.

### Example 2—RNA Associated With TnpB Protein Functions as a Guide Sequence

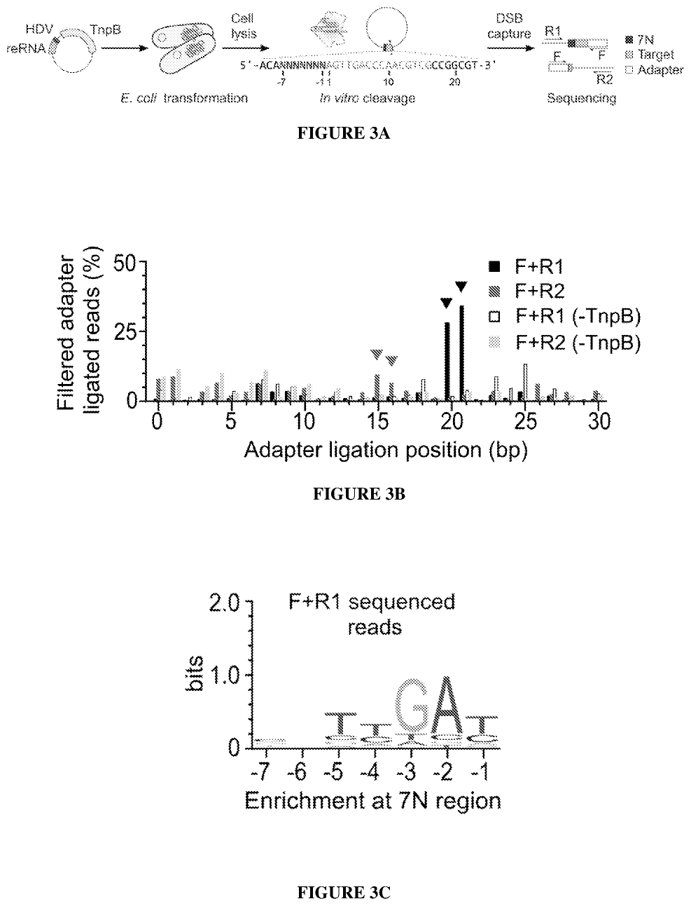

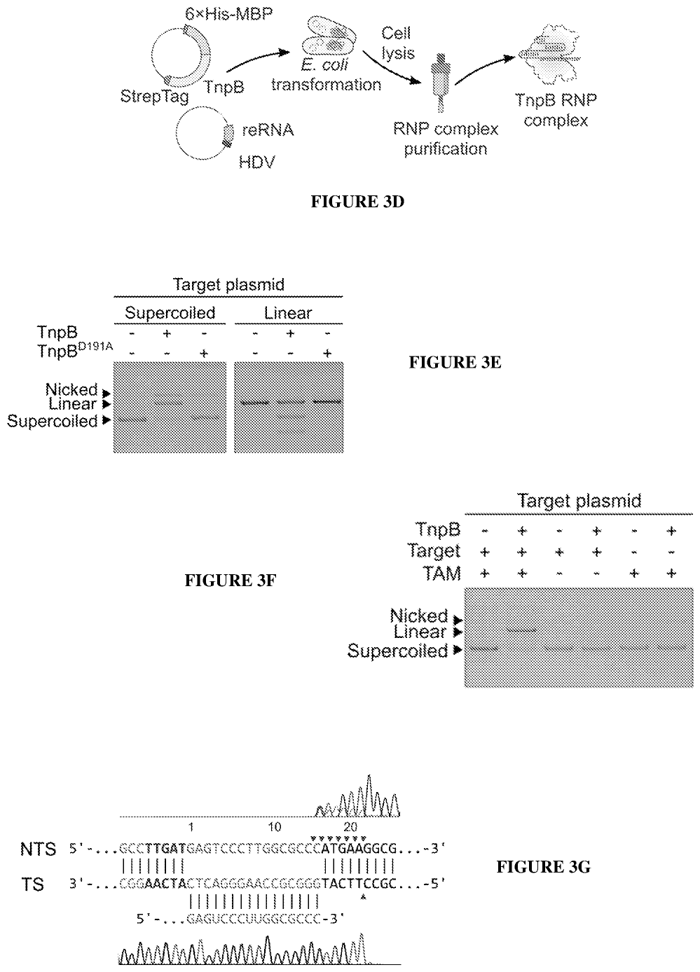

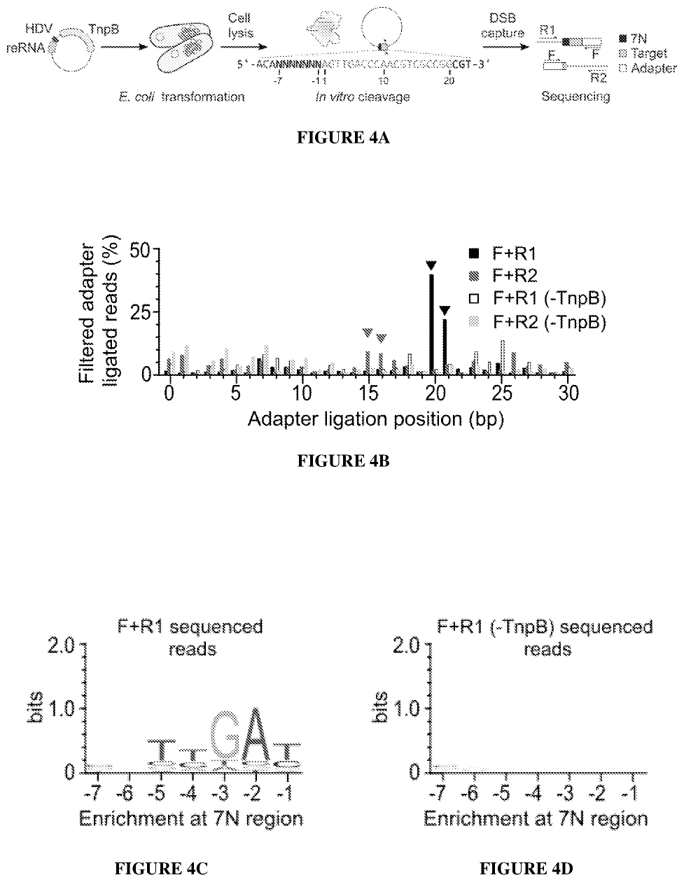

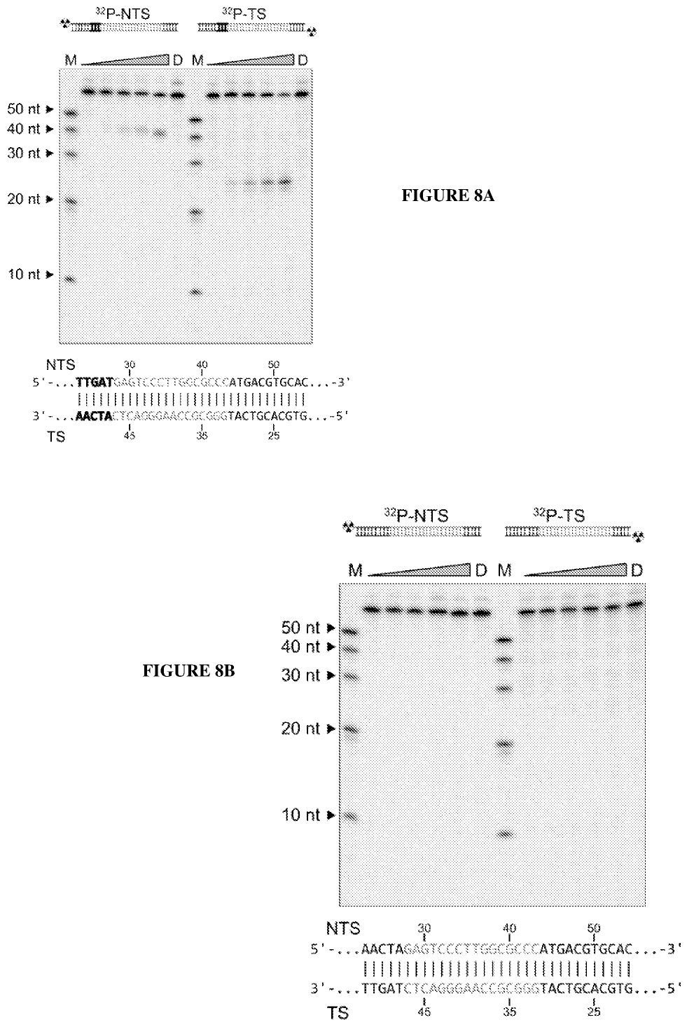

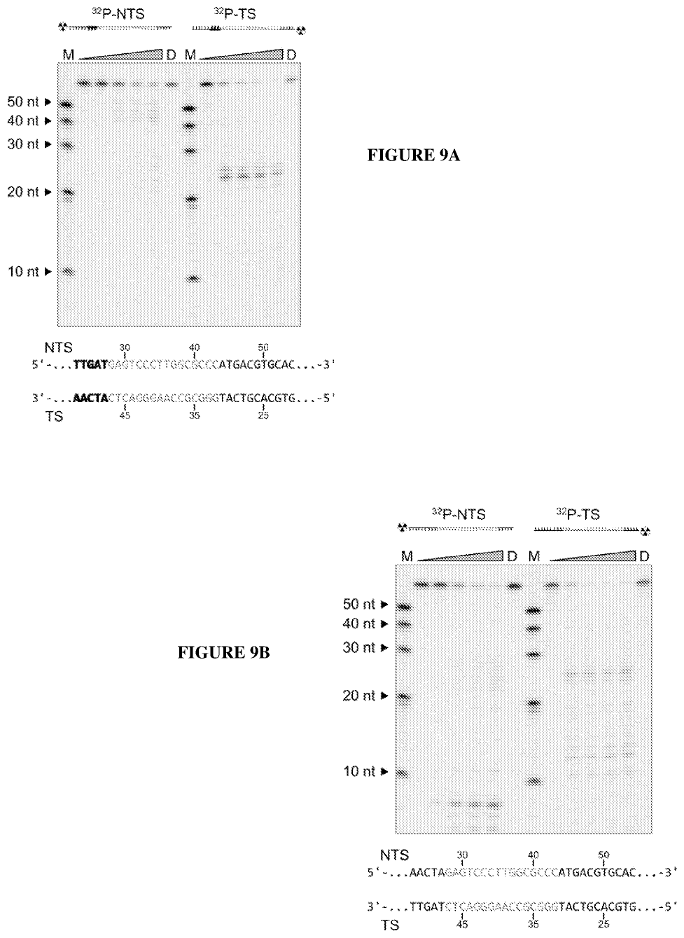

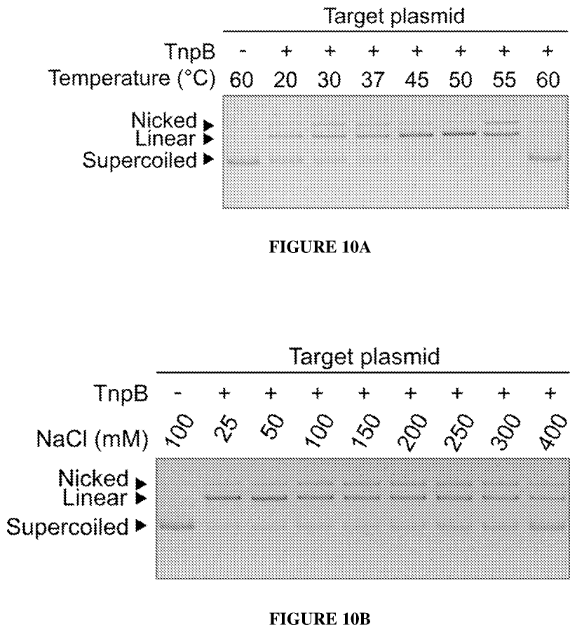

We assumed that the 3′-terminal ˜16 nt of reRNA, which are derived from the DNA adjacent to the transposon and would be variable per se (FIG. 1D), might function as a guide sequence that direct the TnpB to its target and activate DNA cleavage by the RuvC-like active site. To test this hypothesis, we adopted PAM (protospacer adjacent motif) identification assay developed previously for Cas9 and Cas12 nucleases (Karvelis et al., 2015, 2019). In brief, first we engineered reRNA variant where the 3′-terminal TnpB reRNA sequence derived from the plasmid was replaced by 16 nt or 20 nt sequences matching the target next to 7N randomized plasmid library (FIGS. 3A and 4A). Next, following E. coli transformation and expression, cell lysates containing TnpB RNP complexes were used directly to establish randomized plasmid library cleavage. The DNA ends that would result from the plasmid cleavage were repaired by T4 DNA polymerase, subjected for adapter ligation, PCR amplified and sequenced. Analysis of the adapter-ligated fragments revealed the enrichment of the products with adapters at the target site 21-22 bp and 15 bp from the randomized region indicating plasmid library cleavage by TnpB RNP complex (FIGS. 3B and 4B). Analysis of adapter ligation positions for targeted (TS) and non-targeted (NTS) strands suggested staggered cleavage generating 5′-overhangs. Further analysis of DNA fragments revealed enrichment of “TTGAT” sequences in the randomized 7N region 5′-upstream of the target sequence. Notably, the TTGAT sequence which licensed cleavage of plasmid library by TnpB matched the target site sequence required for TnpA mediated ISDra2 transposon excision and insertion (FIGS. 3C, 4C and 4D) (Islam et al., 2003). Since this sequence was analogous to the protospacer adjacent motif (PAM) sequence required for initiation of DNA cleavage by Cas9 or Cas12 nucleases, we termed it Transposon Associated Motif (TAM). Next, to validate the dsDNA cleavage requirements established using plasmid library, we purified the TnpB RNP from E. coli and tested its ability to cleave various dsDNA substrates that contained target sequence flanked by 5′-TTGAT TAM sequence (FIGS. 3D, 8, 9 and 10). TnpB complex cleaved plasmid DNA (both supercoiled and linearized) containing the target flanked by TAM sequence (FIGS. 3E, 4C and 4D). TAM and target sequence matching reRNA guide sequence were required for plasmid DNA cleavage (FIG. 3F). Mutation of the conserved residues in the RuvC-like active site compromised cleavage indicating that RuvC is responsible for dsDNA cleavage (FIG. 3E). Finally, run-off sequencing of the cleavage products confirmed staggered cleavage pattern at 15-21 bp from the TAM resulting in 5′-overhangs (FIGS. 3G and 8). Taken together, these results demonstrate that in vitro TnpB functions as the TAM-dependent RNA-guided dsDNA nuclease.

### Example 3—TnpB is Capable of Cleaving Donor Joint In Vivo

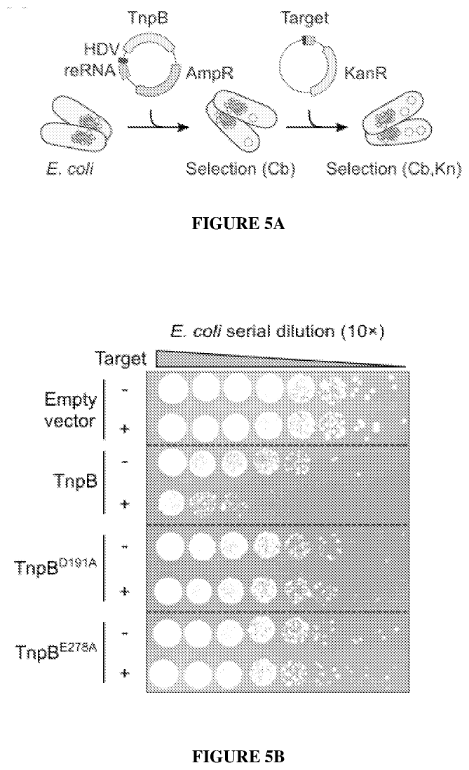

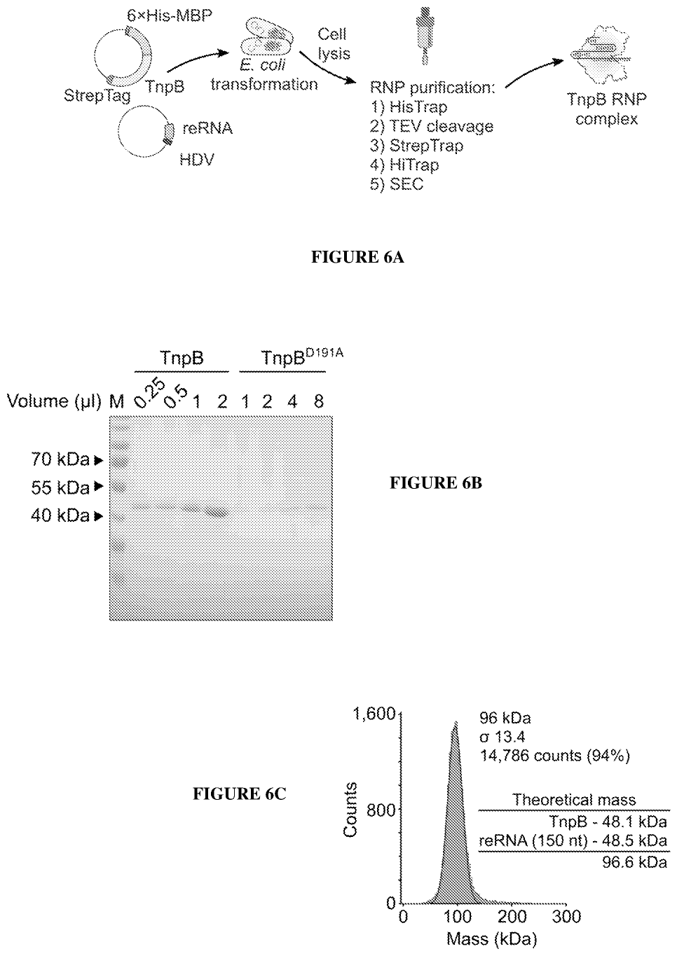

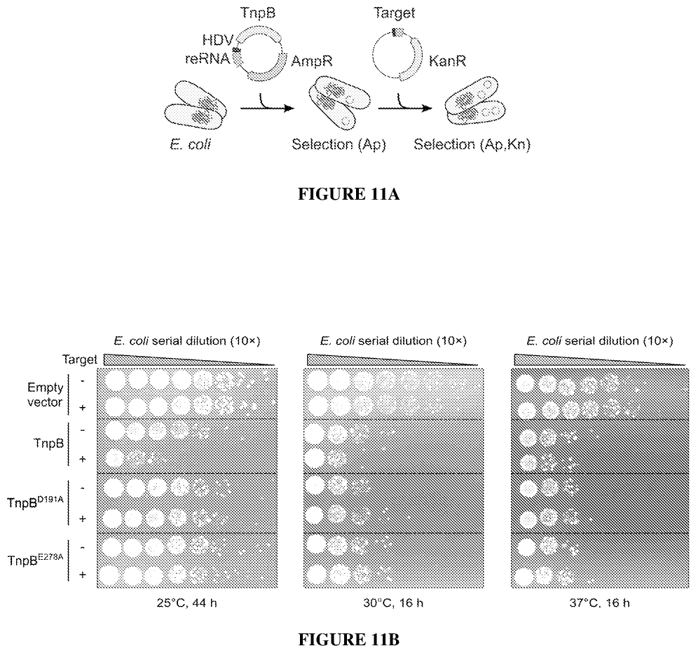

To test whether TnpB is able to generate DSB at the donor joint (FIG. 5A) in the cell we monitored transformation efficiency of recombinant E. coli host expressing TnpB complex by a plasmid containing the TAM flanked target and carrying Kanamycin (Kn) resistance gene that enable growth on Kn supplemented agar plates. Serial dilutions of the transformants revealed plasmid interference in the cells containing TnpB variant with intact RuvC-like active site. Notably, the plasmid interference was more pronounced at lower temperatures (FIG. 5B and FIG. 11). Therefore, these results confirm that TnpB is capable to cleave donor joint in vivo.

### Example 4—TnpB can Mediate Targeted Genome Modification in Cells

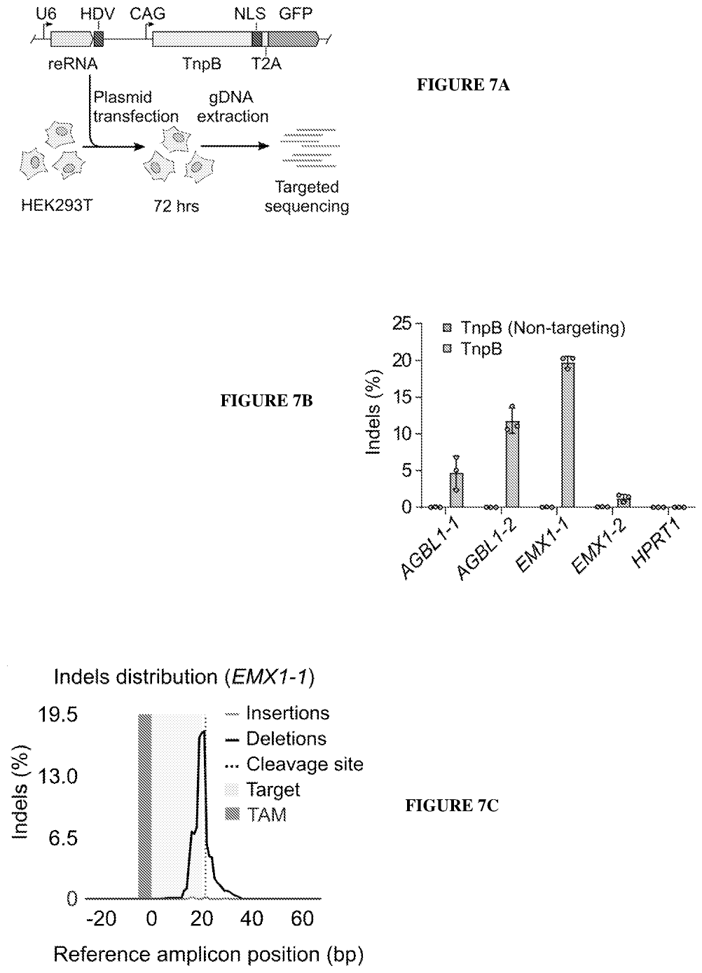

We tested whether TnpB can be adopted for targeted genome modification in human HEK293T cells. Plasmids encoding TnpB protein with nuclear localization sequence (NLS) and reRNA constructs targeting human genomic DNA (gDNA) were transiently transfected into HEK293T cells (FIG. 7A). After 72 h gDNA was extracted and analyzed by sequencing for the presence of insertions and deletions (indels) at the targeted cleavage sites indicating DSB repair events. At the two tested sites (AGBL1-2 and EMX1-1) TnpB introduced mutations at the frequencies of 10-20% (FIG. 7B) similarly to the levels observed for CRISPR-Cas9 and Cas12 based editing (Cong et al., 2013; Jinek et al., 2013; Liu et al., 2019; Mali et al., 2013; Pausch et al., 2020; Zetsche et al., 2015). AGBL1-1 and EMX1-2 sites were moderately (1-5%) modified while no indels were detected at HPRT1 site. Further analysis of the obtained indels revealed dominating deletions at the cleavage site (FIG. 7C) similarly to the mutational profiles generated by Cas12 cleavage (Pausch et al., 2020; Zetsche et al., 2015).

Taken together, these results indicate that extremely compact RNA-guided TnpB nucleases are able to cleave eukaryotic gDNA and can be adopted as the tools for genome editing. providing a new class of extremely compact non-Cas nucleases with different biochemical requirements for genome editing applications. The table below provides a comparison of RNA-guided TnpB nucleases with the Cas9 and Cas12 nucleases.

| RNA-guided | | | |
|---|---|---|---|
| genome editor | Cas 9 | Cas 12 | TnpB |
| System | CRISPR-Cas | CRISPR-Cas | IS200/IS605 |
| | | | and IS607 |
| Protein | 1000-1500 aa | 500-1500 aa | 300-600 aa |
| gRNA | crRNA and | crRNA or | reRNA |
| | tracrRNA | crRNA and | |
| | | tracrRNA | |
| Effector complex | 1:1 | 1:1 or 2:1 | 1:1 |
| (protein:gRNA) | | (Cas12f) | |
| Nuclease active site | HNH and RuvC | RuvC | RuvC |
| dsDNA target | Target and 3′ | 5′ PAM and | 5′ TAM and |
| | PAM | target | target |

The examples described herein are to be understood as illustrative examples of embodiments of the invention. Further embodiments and examples are envisaged. Any feature described in relation to any one example or embodiment may be used alone or in combination with other features. In addition, any feature described in relation to any one example or embodiment may also be used in combination with one or more features of any other of the examples or embodiments, or any combination of any other of the examples or embodiments. Furthermore, equivalents and modifications not described herein may also be employed within the scope of the invention, which is defined in the claims.

All publications referred to herein are incorporated by reference in their entirety to the same extent as if each individual publication was specifically and individually indicated to be incorporated by reference in its entirety.

### REFERENCES

1. Anzalone et al., (2020) Genome editing with CRISPR-Cas nucleases, base editors, transposases and prime editors. Nature Biotechnology 38, 824-844
2. Barabas, O., Ronning, D. R., Guynet, C., Hickman, A. B., Ton-Hoang, B., Chandler, M., and Dyda, F. (2008). Mechanism of IS200/IS605 Family DNA Transposases: Activation and Transposon-Directed Target Site Selection. Cell 132, 208-220.
3. Boocock, M. R., and Rice, P. A. (2013). A proposed mechanism for IS607-family serine transposases. Mob DNA 4, 24.
4. Chen, W., Mandali, S., Hancock, S. P., Kumar, P., Collazo, M., Cascio, D., and Johnson, R. C. (2018). Multiple serine transposase dimers assemble the transposon-end synaptic complex during IS607-family transposition. ELife 7, e39611.
5. Clement, K., Rees, H., Canver, M. C., Gehrke, J. M., Farouni, R., Hsu, J. Y., Cole, M. A., Liu, D. R., Joung, J. K., Bauer, D. E., et al. (2019). CRISPResso2 provides accurate and rapid genome editing sequence analysis. Nature Biotechnology 37, 224-226.
6. Cong, L., Ran, F. A., Cox, D., Lin, S., Barretto, R., Habib, N., Hsu, P. D., Wu, X., Jiang, W., Marraffini, L. A., et al. (2013). Multiplex Genome Engineering Using CRISPR/Cas Systems. Science 339, 819-823.
7. Crooks, G. E. (2004). WebLogo: A Sequence Logo Generator. Genome Research 14, 1188-1190.
8. Gillmore et al., (2021) CRISPR-Cas9 in vivo gene-editing for transthyretin amyloidosis. NEJM DOI: 10.1056/NEJMoa2107454, 26 Jun. 2021
9. Gomes-Filho, J. V., Zaramela, L. S., Italiani, V. C. da S., Baliga, N. S., Vêncio, R. Z. N., and Koide, T. (2015). Sense overlapping transcripts in IS1341-type transposase genes are functional non-coding RNAs in archaea. RNA Biol 12, 490-500.
10. Guynet, C., Hickman, A. B., Barabas, O., Dyda, F., Chandler, M., and Ton-Hoang, B. (2008). In Vitro Reconstitution of a Single-Stranded Transposition Mechanism of IS608. Molecular Cell 29, 302-312.
11. Hajian et al., (2019) Detection of unamplified target genes via CRISPR-Cas9 immobilized on a graphene field-effect transistor. Nature Biomedical Engineering 3, 427-437
12. He, S., Hickman, A. B., Dyda, F., Johnson, N. P., Chandler, M., and Ton-Hoang, B. (2011). Reconstitution of a functional IS608 single-strand transpososome: role of non-canonical base pairing. Nucleic Acids Research 39, 8503-8512.
13. Hickman, A. B., Chandler, M., Dyda. F. (2010) Integrating prokaryotes and eukaryotes: DNA transposases in light of structure. Crit. Rev. Biochem. Mol. Biol. 45, 50-56.
14. Hoang, B. T., Pasternak, C., Siguier, P., Guynet, C., Hickman, A. B., Dyda, F., Sommer, S., and Chandler, M. (2010). Single-stranded DNA transposition is coupled to host replication. Cell 142, 398-408.
15. Islam, M. S., Hua, Y., Ohba, H., Satoh, K., Kikuchi, M., Yanagisawa, T., and Narumi, I. (2003). Characterization and distribution of IS8301 in the radioresistant bacterium Deinococcus radiodurans. Genes Genet. Syst. 78, 319-327.
16. Jiang, F., and Doudna, J. (2017). CRISPR-Cas9 Structures and Mechanisms. Ann. Rev. Biophys. 46, 505-29.
17. Jinek, M., East, A., Cheng, A., Lin, S., Ma, E., and Doudna, J. (2013). RNA-programmed genome editing in human cells. ELife 2, e00471.
18. Kapitonov, V. V., Makarova, K. S., and Koonin, E. V. (2016). ISC, a Novel Group of Bacterial and Archaeal DNA Transposons That Encode Cas9 Homologs. J. Bacteriol. 198, 797-807.
19. Karvelis, T., Gasiunas, G., Young, J., Bigelyte, G., Silanskas, A., Cigan, M., and Siksnys, V. (2015). Rapid characterization of CRISPR-Cas9 protospacer adjacent motif sequence elements. Genome Biol 16, 253.
20. Karvelis, T., Young, J. K., and Siksnys, V. (2019). A pipeline for characterization of novel Cas9 orthologs. In Methods in Enzymology, (Elsevier). pp. 219-240.
21. Karvelis, T., Bigelyte, G., Young, J. K., Hou, Z., Zedaveinyte, R., Budre, K., Paulraj, S., Djukanovic, V., Gasior, S., Silanskas, A., et al. (2020). PAM recognition by miniature CRISPR-Cas12f nucleases triggers programmable double-stranded DNA target cleavage. Nucleic Acids Res 48, 5016-5023.
22. Kersulyte, D., Mukhopadhyay, A. K., Shirai, M., Nakazawa, T., and Berg, D. E. (2000). Functional Organization and Insertion Specificity of IS607, a Chimeric Element of Helicobacter pylori. Journal of Bacteriology 182, 5300-5308.
23. Kersulyte, D., Velapatiño, B., Dailide, G., Mukhopadhyay, A. K., Ito, Y., Cahuayme, L., Parkinson, A. J., Gilman, R. H., and Berg, D. E. (2002). Transposable Element ISHp608 of Helicobacter pylori: Nonrandom Geographic Distribution, Functional Organization, and Insertion Specificity. Journal of Bacteriology 184, 992-1002.
24. Knott et al., (2018) CRISPR-Cas guides the future of genetic engineering. Science 361, 866-869
25. Krupovic, M., Makarova, K. S., Forterre, P., Prangishvili, D., and Koonin, E. V. (2014). Casposons: a new superfamily of self-synthesizing DNA transposons at the origin of prokaryotic CRISPR-Cas immunity. BMC Biology 12, 36.
26. Li, H., and Durbin, R. (2009). Fast and accurate short read alignment with Burrows-Wheeler transform. Bioinformatics 25, 1754-1760.
27. Li, H., Handsaker, B., Wysoker, A., Fennell, T., Ruan, J., Homer, N., Marth, G., Abecasis, G., Durbin, R., and 1000 Genome Project Data Processing Subgroup (2009). The Sequence Alignment/Map format and SAMtools. Bioinformatics 25, 2078-2079.
28. Liu, J.-J., Orlova, N., Oakes, B. L., Ma, E., Spinner, H. B., Baney, K. L. M., Chuck, J., Tan, D., Knott, G. J., Harrington, L. B., et al. (2019). CasX enzymes comprise a distinct family of RNA-guided genome editors. Nature 566, 218-223.
29. Ma H, Tu L C, Naseri A, Chung Y C, Grunwald D, Zhang S, Pederson T., (2018) CRISPR-Sirius: RNA scaffolds for signal amplification in genome imaging. Nat Methods. November; 15(11):928-931.
30. Ma H, Tu L C, Naseri A, Huisman M, Zhang S, Grunwald D, Pederson T. (2016) Multiplexed labeling of genomic loci with dCas9 and engineered sgRNAs using CRISPRainbow. Nat Biotechnol. May; 34(5):528-30.
31. Madeira, F., Park, Y. M., Lee, J., Buso, N., Gur, T., Madhusoodanan, N., Basutkar, P., Tivey, A. R. N., Potter, S. C., Finn, R. D., et al. (2019). The EMBL-EBI search and sequence analysis tools APIs in 2019. Nucleic Acids Res 47, W636-W641.
32. Makarova, K. S., Wolf, Y. I., Iranzo, J., Shmakov, S. A., Alkhnbashi, O. S., Brouns, S. J. J., Charpentier, E., Cheng, D., Haft, D. H., Horvath, P., et al. (2020). Evolutionary classification of CRISPR-Cas systems: a burst of class 2 and derived variants. Nat Rev Microbiol 18, 67-83.
33. Mali, P., Yang, L., Esvelt, K. M., Aach, J., Guell, M., DiCarlo, J. E., Norville, J. E., and Church, G. M. (2013). RNA-Guided Human Genome Engineering via Cas9. Science 339, 823-826.
34. Maresca et al., (2013). Obligate Ligation-Gated Recombination (ObLiFaRe): Custom-designed nuclease-mediated targeted integration through nonhomologous end joining. Genome Research 23, 539-546.
35. Martin, M. (2011). Cutadapt removes adapter sequences from high-throughput sequencing reads. EMBnet. Journal 17, 10-12.
36. Mir et al., Heavily and fully modified RNAs guide efficient SpyCas9-mediated genome editing—Nature Communications, 9, Article No. 2641 (2018).
37. Pasternak, C., Ton-Hoang, B., Coste, G., Bailone, A., Chandler, M., and Sommer, S. (2010). Irradiation-Induced Deinococcus radiodurans Genome Fragmentation Triggers Transposition of a Single Resident Insertion Sequence. PLOS Genet 6, e1000799.
38. Pasternak, C., Dulermo, R., Ton-Hoang, B., Debuchy, R., Siguier, P., Coste, G., Chandler, M., and Sommer, S. (2013). ISDra2 transposition in Deinococcus radiodurans is downregulated by TnpB. Molecular Microbiology 88, 443-455.
39. Pausch, P., Al-Shayeb, B., Bisom-Rapp, E., Tsuchida, C. A., Li, Z., Cress, B. F., Knott, G. J., Jacobsen, S. E., Banfield, J. F., and Doudna, J. A. (2020). CRISPR-CasΦ from huge phages is a hypercompact genome editor. Science 369, 333-337.
40. Robinson, J. T., Thorvaldsdóttir, H., Winckler, W., Guttman, M., Lander, E. S., Getz, G., and Mesirov, J. P. (2011). Integrative genomics viewer. Nature Biotechnology 29, 24-26.
41. Sajwan S, Mannervik M (2019) Gene activation by dCas9-CBP and the SAM system differ in target preference. Scientific Reports 9: Article No. 18104
42. Shmakov, S., Smargon, A., Scott, D., Cox, D., Pyzocha, N., Yan, W., Abudayyeh, O. O., Gootenberg, J. S., Makarova, K. S., Wolf, Y. I., et al. (2017). Diversity and evolution of class 2 CRISPR-Cas systems. Nat Rev Microbiol 15, 169-182.
43. Siguier, P., Perochon, J., Lestrade, L., Mahillon, J., and Chandler, M. (2006). ISfinder: the reference centre for bacterial insertion sequences. Nucleic Acids Res 34, D32-36.
44. Siguier, P., Gourbeyre, E., and Chandler, M. (2014). Bacterial insertion sequences: their genomic impact and diversity. FEMS Microbiology Reviews 38, 865-891.
45. Takeda, S. N., Nakagawa, R., Okazaki, S., Hirano, H., Kobayashi, K., Kusakizako, T., Nishizawa, T., Yamashita, K., Nishimasu, H., and Nureki, O. (2020). Structure of the miniature type V-F CRISPR-Cas effector enzyme. Molecular Cell S1097276520308352.
46. Xiao, R., Li, Z., Wang, S., Han, R., and Chang, L. (2021). Structural basis for substrate recognition and cleavage by the dimerization-dependent CRISPR-Cas12f nuclease. Nucleic Acids Research.
47. Xu et al., (2019) Viral Delivery Systems for CRISPR. Viruses, 11, No. 28
48. Zetsche, B., Gootenberg, J. S., Abudayyeh, O. O., Slaymaker, I. M., Makarova, K. S., Essletzbichler, P., Volz, S. E., Joung, J., van der Oost, J., Regev, A., et al. (2015). Cpf1 Is a Single RNA-Guided Endonuclease of a Class 2 CRISPR-Cas System. Cell 163, 759-771.

1-200. (canceled)

201. A method for cleaving a polynucleotide with an effector complex, wherein the polynucleotide comprises a target sequence, the effector complex comprising:
(a) a protein comprising or consisting of a TnpB protein; and
(b) an RNA comprising:
(i) a polynucleotide-targeting segment comprising a guide sequence capable of hybridising to the target sequence; and
(ii) a protein-binding segment that allows the RNA to bind the TnpB protein to form the effector complex,
wherein the method comprises contacting the polynucleotide with the effector complex and allowing the TnpB protein to cleave the polynucleotide.

202. The method according to claim 201, wherein the TnpB protein has an amino acid sequence of a protein obtained from a tnpB gene of a mobile genetic element in the IS200/IS605 or the IS607 families.

203. The method according to claim 202, wherein the TnpB protein has an amino acid sequence of a protein obtained from a tnpB gene of a mobile genetic element from the IS200/IS605 family.

204. The method according to claim 203, wherein the TnpB protein has an amino acid sequence of a protein obtained from a tnpB gene of the mobile genetic element ISDra2 from Deinococcus radiodurans.

205. The method according to claim 204, wherein the TnpB protein has at least 85% sequence identity with the amino acid sequence of the protein obtained from the tnpB gene.

206. The method according to claim 205, wherein the TnpB protein comprises or consists of the amino acid sequence of SEQ ID NO: 1, or wherein the TnpB protein comprises or consists of an amino acid sequence having at least 85% sequence identity with SEQ ID NO: 1.

207. The method according to claim 201, wherein the RNA is 50 to 300 nucleotides in length.

208. The method according to claim 201, wherein the guide sequence of the RNA is 10 to 30 nucleotides in length.

209. The method according to claim 201, wherein the protein-binding segment of the RNA comprises a right end of insertion sequence of the IS200/IS605 or the IS607 family and a part of the sequence encoding the TnpB protein.

210. The method according to claim 201, wherein the protein-binding segment comprises a sequence comprising SEQ ID NO: 16 or a sequence having at least 85% sequence identity with SEQ ID NO:16.

211. The method according to claim 201, wherein the polynucleotide comprises a Transposon Associated Motif, and wherein Transposon Associated Motif of the polynucleotide is TTGAT.

212. The method according to claim 211, wherein the TnpB protein comprises or consists of the amino acid sequence of SEQ ID NO: 1, or an amino acid sequence having at least 85% sequence identity with SEQ ID NO:1, and wherein the protein-binding segment of the RNA comprises SEQ ID NO: 16 or a sequence having at least 85% sequence identity with SEQ ID NO:16.

213. An effector complex for binding to a target region in a polynucleotide, the effector complex comprising a protein and an RNA, wherein the protein comprises or consists of a TnpB protein, and wherein the RNA comprises:
(i) a polynucleotide-targeting segment comprising a guide sequence that is capable of hybridising with a target sequence that is comprised in the target region; and
(ii) a protein-binding segment that binds to the TnpB protein.

214. The effector complex according to claim 213, wherein the TnpB protein has an amino acid sequence of a protein obtained from a tnpB gene of a mobile genetic element in the IS200/IS605 or the IS607 families or is a variant thereof having an amino acid sequence that has at least 85% sequence identity therewith.

215. The effector complex according to claim 213, wherein the effector complex comprises a TnpB protein with an inactivated nuclease domain.

216. The effector complex of claim 213, which comprises one or more effector molecules for modification of the target region.

217. The effector complex of claim 216, wherein the one or more effector molecules are selected from an endonuclease that is not the TnpB protein, a ribonuclease, a nickase, a base editor, an epigenetic modifier, a transposase, a recombinase, and a reverse transcriptase.

218. The effector complex of claim 216, wherein the protein is a fusion protein comprising the TnpB protein and the one or more effector molecules.

219. A mutated TnpB protein comprising a mutation to inactivate the nuclease domain of the protein, wherein the TnpB protein is configured to bind to an RNA for guiding an effector complex to a target region in a polynucleotide, the RNA comprising:
(ii) a polynucleotide-targeting segment comprising a guide sequence capable of hybridising to a target sequence in the target region of the polynucleotide; and
(ii) a protein-binding segment that allows the RNA to bind to a TnpB protein to form the effector complex.

220. A method for modifying, labelling or controlling expression from a target region in a polynucleotide with an effector complex, wherein the target region comprises a target sequence,
wherein the effector complex is an effector complex of claim 213,
wherein the method comprises contacting the polynucleotide with the effector complex such that the guide sequence of the RNA hybridises to the target sequence, allowing the effector complex to modify or label the target region or control expression from the target region.


SEQUENCE LISTING (19 sequences)

SEQ ID NO: 1 - protein, 408 aa, Deinococcus radiodurans
MIRNKAFVVRLYPNAAQTELINRTLGSARFVYNHFLARRIAAYKESGKGLTYGQTSSELTLLKQAEETSWLSEVDKFALQNSLKNLETAYKNFFRTVKQSGKKVGFPRFRKKRTGESYRTQFTNNNIQIGEGRLKLPKLGWVKTKGQQDIQGKILNVTVRRIHEGHYEASVLCEVEIPYLPAAPKFAAGVDVGIKDFAIVTDGVRFKHEQNPKYYRSTLKRLRKAQQTLSRRKKGSARYGKAKTKLARIHKRIVNKRQDFLHKLTTSLVREYEIIGTEHLKPDNMRKNRRLALSISDAGWGEFIRQLEYKAAWYGRLVSKVSPYFPSSQLCHDCGFKNPEVKNLAVRTWTCPNCGETHDRDENAALNIRREALVAAGISDTLNAHGGYVRPASAGNGLRSENHATLVV

SEQ ID NO: 2 - protein, 383 aa, Helicobacter pylori
MLITYKQKLYKNDKNRRIDTLLRRYGALYNHCIALHKRYYRLFKKYLKLYDLQKHITKLKKTHRYAFLKTLGSQTMQDLTERIDKAFKKFFNKKAKLPRFKKVANYKSFTFKSKIDKKTGLNKGVGFAIKDNVVSFNGYSYKFIKTYAFIGKVKTLTIKRDNTGDYFLCLVCELENHPNKQTACDKSVGFDFGLKTFLTGSDHTKIESPLSFSKYLPLIKRLSKNLSKKVKGSNNFKKAKKKLTQLHQKIKYLRTDFFHKLALKLSREYQTIFIEDLNMKAMQKLWGRKVSDLAFSEFVKILENKANVVKIDRFYPSSKTCSNCLFVNEEINKDFRKIGKTDKEREYHCKYCGLELDRDLNAAINIHRVGASTLGVEFVRPTC

SEQ ID NO: 3 - protein, 427 aa, Helicobacter pylori
MLNAIKFRIYPNAQQKELISKHFGCSRVVYNYFLDYRQKQYAKGIKETYFTMQKVLTQIKHQEKYHYLNECNSQSLQMALRQLVSAYDNFFSKRARYPKFKSKKNAKQSFAIPQNIEIKTETQTIALPKFKEGIKAKLHRELPKDSVIKQAFISCIADQYFCSISYETKEPIPKPTIIKKAVGLDMGLRTLIVTSDKIEYPHIRFYQKLEKKLTKAQRRLSKKVKGSNNRKKQAKKVARLHLACSNTRDDYLHKISNEITNQYDLIGVETLNVKGLMRTYHSKSLANASWGKFLTMLKYKAQRKAKTLLGIDRFFPSSQLCSYCGFNTGKKHENITKFTCPHCNITHHRDYNASVNIRNYALGMLDDRHKIKIDKSRVGIIRTDYAHYTDERIKACGASSNGVISKYGNILDLASYGAMKQEKAQSL

SEQ ID NO: 4 - protein, 442 aa, Helicobacter pylori
MKVNKGFKFRLYPTKEQQDKLQRCFFVYNQAYNIGLNLLQEQYETNKDSPPKERKWKKSSELDKAIKHHLNARGLSFSSVIAQQSRMNVERALKDAFKVKDRGFPKFKNSKSAKQSFSWNNQGFSIKDSDEERFKIFTLMKMPLMMRMHRDFPPHSKVKQIVISWSHRKYFVSFCVEYEQDITPIKNPKNGVGLDLNILDIACSCGVNNHKKLTDFKQYPTDMKELLGIEIDEELDTKRLIPTYSKLYSLKKYSKKFKRLQRKQSRRVLKSKQNKTKLGGNFYKTQKKLNQAFDKSSHQKTDRYHKITSELSKQFELVVVEDLQVKNMTKRAKLKNVKQKSGLNQSILNTSFYQIISFLDYKQQHNGKLLVKVPPQYTSKTCHCCGNINHKLKLNHRQYWCLECGYREHRDINAANNIISKGLSLFGVGNIHADFKEQSLSC

SEQ ID NO: 5 - protein, 402 aa, Escherichia coli
MKRLQAFKFQLRPGGQQEREMRRFAGACRFVFNRALALQNENHEAGNKYIPYGKMASWLVEWKNATETQWLKDAPSQPLQQSLKDLERAYKNFFRKRAAFPRFKKRGQNDAFRYPQGVKLDQENSRIFLPKLGWMRYRNSRQVTGVVKNVTASQSCGKWYISIQTENEVSTPVHPSALMVGLDAGVAKLATLSDGTVFGPVNSFQKNQKTLARLQRQLSRKVKFSNNWQKQKRKIQRLHSCIANICRDYLHKVTTTVSKNHAMIVIEDLKVSNMSKSAAGTVSQPGRNVRAKSGLNRSILDQGWYEMRRQLEYKQLWRGGQVLAVPPAYTSQRCACCGHTAKENRLSQSKFRCQACGYTANADVNGARNILAAGHAVLACGEMVQSGRPLKQEPTEMIQATA

SEQ ID NO: 6 - protein, 369 aa, Thermophilic bacterium
MANKAYQFRLYPTKEQEQLLAKTFGCVRFVYNKMLEERIQMFEKFKDDQESLKQQTCPTPAKYKKEFPWLKEVDSLALANAQLNLQKAFQHFFSGRAGFPKFKNRKAKQSYTTNMVNGNIKLSDGYIKLPKLKWIKLKQHREIPAHHIIKSCTITKTKTGKYYISILTEYEHQPAPKEVQTVVGLDFSMSTLYVDSEGKRANYPRFYRKALETLAKEQRKWSRKKKGSNRWHKQRLKVAKLHEKIANQRKDFLHKESHKLAKRYDCVVIEDLNMKGMSQALHFGQGVHDNGWGMFTTFLQYKLVEQGKKLIKIDKWFPSSKTCSCCGRVKESLSLSERTFRCECGFESDRDVNAAINIKHEGMKRLAIV

SEQ ID NO: 7 - protein, 393 aa, Sulfolobus solfataricus
MPTLGFRFRAYTDEQTLRALKAQLKLTCEIYNTLRWADIYFYQRDGKGLTQTELRQLALDLRKQDDEYKQLYSQVVQQVADRYSEAKKRFFEGLARFPKEKKPHKYYSLVYTQSGWKILHVREIRKGKKNKKKLITLKLSNLGTFKVIVHRDFPLDKVKRVVVKLTRSERIYITFVVDHEFPKLPNTGKVVAIDVGVEKLLITSDGEYFPNLRPYEKALWKVKHIHRELSRKKFLSNNWFKAKVKLARAYEHLKNLRTDLYMKLGKWFAEHYDVVVMEGIHAKQLVGKSLRSLRRRLSDVGFGELRGVLKYQLEKYGKKLILVNPAYTSKTCARCGYVKNDLSLSDRVFVCPNCGWIADRDYNASLNILRGSGSERPLVWSSALYQYSGKVGL

SEQ ID NO: 8 - protein, 401 aa, Anabaena sp.
MLVFETKLEGTNEQYQLLMRRLKLLVLSNACLRTWIGQPNIGRYDLSAYCAVLLPMKTFRSLPNSTLWLDKLLLKERGVQLLGFLTIASKTKPGRKVIHALKKNRRMGVLSIKLAAGSLVVTVAYVTFSDGFKAGTFKLWGTRDLHFYQLKQFKRVRVVRRADGYYAQFCIDQERVERREPTLKTIGLDVGLNHFLTDSEGNTVENPRHLRKSEKSLKRLQRRLSKTKKGSNNRVKARNRLSRKHLKVSRQRKDFAVKLARCVVQSSDLVAYEDLQVRNMVRNRHLAKSISDAAWTQFRQWVEYFGKVFGVVTVAVPPHHTSQNCSNCGEVVKKSLSTRTHACPHCGHIQDRDWNAARNILELGLRTVGHTGSQVSGDIDLCLGEVTPPNKSSRGKRKPKK

SEQ ID NO: 9 - protein, 376 aa, Escherichia coli
MKRAYKYRFYPTTEQAELLAQTFGCVRFVYNSILRWRTDAYYERKEKIGYLQANARLTALKKEPEYIWLNDVSCVPLQQSLRHQQAAFANFFAGRAAYPAFKSKRHKQVAEFTASAFKHRDGELYIAKSKSPLDVRWSRELPSAPSTVTISRDSAGRYFVSCLCEFEPVSMPVTAKTVGIDVGLKDLFVTDTGFKTDNPRHTAKYAKRLTLLQRRLSRKQKGSRNRIKARLKVARLHAKIADCRMDNLHKLSRKLINENQVVCVESLKVKNMIRNPKLSKAIADAGWSELVRQLQYKGKWAGRSVVAIDQYLPSSKCCSCCGFTMQKMPLNVRKWHCPECGADHDRDINAARNIKAAGLAVLAHGEPVNPESQHAA

SEQ ID NO: 10 - protein, 393 aa, Thermosynechococcus elongatus
MRGVEKAFSYRFYPTTEQESLLRKTLGCVRLVYNRALAARTEAWYERKERLDYVQTSALLTQWKKQDDLQFLNEVSCVPLQQALRHLQSAFTNFFAGRAKYPNFKKKRNGGSAEFTKSAFRWKDGKVFLAKCNEPLNIPWSRRLPDGVEPSTVTIRLNPAGQWYISLRFDDPRELTLQPVDPSVGLDVGMSSLITLSTGEKIANPKHFNRYYKRLRKAQRSLSRKQKGSRNWDKARLKVAKIHQKISDSRKDHLHQLTTRLIRENQTIIIESLAVKNMVKNRQLARSISDAGWGELVRQLEYKAQWYGRTLVKIDRWFPSSKRCGQCGHIVEWLPLSVREWDCPKCGAHHDRDINAAGNILAVGHTVTVCGAGVRPDRHTSGGQLRRNRKSQK

SEQ ID NO: 11 - protein, 419 aa, Helicobacter pylori
MSAISITHKIALKPNNKHITYFKKAFGCARFAYNWGLAKWKENYQLGIKTSHLQLKKEFNALKKSQFNFVYEVTKYATQQPFIHLNLAFNKFFRDLEKGLVSYPKFKKKREFQGSFYIGGDQIKIIQTANTDYLKIPNLPPIKLTEKLRFQGKIHNATITQKGDHFYVSISCDIDESEYKRTHKLQESHNKLGIDIGIKSFVSLSNGLNIYAPKPLDKLTRKLVRISRQLSKKIHPKTKGDKTRKSNNYLKHSKKLTHLHEKIANIRLDFLHKLTSSLIRHSNSFCLESLKVKNMFKNHRLAKSLSDISMSVFNTLLEYKAKYSNKEILRADTYYPSSKTCSNCQKVKQDLKLKDRIYQCLECGFELDRDINAAINLLKHLVGRVTAEFTPMDLTALLNDLSNNRLATSKVELGIQQKS

SEQ ID NO: 12 - protein, 393 aa, Thermococcus sibiricus
MPSETIKLASKFKLKETPEGLNELFSTYRDIVNFLITHAFENNITSFYRLKKEIYKSLRKEYPELPSHYIYTACQMAASIYKSYRKRKRRGKASGRPVFKKEAIMLDDHLFKLDLEKGIIKLSTPNGRITLKFYPAKHHEKFKNWKVGQAWLVRTPKGVFINVVFSKEVEVKEPEDFVGVDLNENNVTLSLSDGEFVQIITHEKEIRTGYFVKRRKIQKKVKVGKKRQELLEKYGERERNRLNDLYHKLANKIVELAEKYGGIALEDLTEIRNSIRYSAEMNGRLHRWSFRKLQSIIEYKAKLKGVEVVFVDPAYTSSLCPVCGEKLSPNGHRVLKCLNCGFEADRDVVGSWNVRLRALKMWGVSVPPESPPMKMGGGKASRGDVYELYTNYG

SEQ ID NO: 13 - protein, 550 aa, Mycobacterium tuberculosis
MIVRMRSCAQAAKVAEATGGVQLAGKPKPDGTPTFSRYVEIGVDFEAHRPVVESVSVLFELYDGDANSYAATGGPGAQLPSGWMVTAAKFEVEWPADPQRAGLVRSHFGARRKAFNWGLAQVKADLDAKAADPAHESVDWDLKSLRWAWNRAKDDVAPWWAENSKECYSSGLADLAQGLANWKAGKNGTRKGRRVGFPRFKSGRRDPGRVRFTTGTMRIEDDRRTITVPVIGPLRAKENTRRVQRHLVSGRAQILNMTLSQRWGRLFVAVCYALRTPTTRSPLTQPTVRAGMDLGVRTLATVATLDTATGEQTIIEYPNPAPLKATLVARRRAGRELSRRIPGSHGHRAVKAKLARLDRRCVHLRREAAHQLTTELAGTYGQVVIEDLDVAAMKRSMRRRAFRRSVSDAAMGLVAPQLAYKTAKCSGVLTVADRWFASSQIHHGCTSPDGTPCRLQGKGRIDKHLLCPVTGEVVDRDRNAALNLRDWPDNASRGPVGTTAPSAPGPTTTVGTGHGADTGSSGAGGASVRPRPRRAGRGEAKTQTPQGDAA

SEQ ID NO: 14 - protein, 440 aa, Bifidobacterium longum
MSAYEAVRIRLDPTPRQTRLLESHAGGARFAYNLMLAHVRRQISLGEKPDWTLYAMRRWWNEWKDEIAPWWRENSKEAYGSAFEWLSQALRNWSDSRKGRRAGRRVGWPKYKSKRSSVPRFAYTTGSFGLIEDDPKALRLPRIGRVHCMENATERVHGRRIVRMTVSRHAGFWYAALTVERPTESVPAKNRKRKNHDRQVGVDLGVRTLATLSDGTTFPNPRNYVRTQRKLRHAQQSLSRRDRGMSHGCGSKRYNRALERVRRIHARIAAQRADNIGKLTTWLADNYSDISIEDLNVQGMSHNRRLAKHILDADFHEFRRQLEYKTARAGTRLHVIDRWYPSSKTCSNCGTVKAKLSLSERVYHCEECGLVIDRDVNAAINIQVAGSAPETLNARGGSVGQTRLECGTMRHPAKREPSGGDSRVRLGAGLGNEAMQMTSL

SEQ ID NO: 15 - protein, 412 aa, Sulfolobus sp.
MERTIKLRVRVDYITYSALKEVEGEYREVLEDAINYGLSNKTTSFTRIKAGVYKTEREKHKDLPSHYIYTACEDASERLDSFEKLKKRGRSYTEKPSVRKVTVHLDDHLWKFSLDKISISTMQGRVFISPTFPKIFWRYYNTEWRIASEARFKLLKGNVVEFFIVFKRDEPKPYEPKGFIPVDLNEDSVSVLVDGKPMLLETNTKRITLGYEYRRKAITTRRSAEDREVKRKLKRLRERDKKVVIRRKLAKLIVKEAFESMSAIVLEALPRRPPEHMIKDVKDSQLRLRIYRSAFSSMKNAIIEKAKEFRVPVVLVNPSYTSSTCPIHGAKIVYQPDGGDAPRVGVCEKGKEKWHRDVVALYNLRKRAGDVSPVPLGSKESHDPPTVKLGRWLRAKSLHSIMNEHKMIEMKV

SEQ ID NO: 16 - RNA, 32 nt, Deinococcus radiodurans
gaaucacgcgacuuuagucgugugagguucaa

SEQ ID NO: 17 - DNA, 16 bp, Artificial Sequence
  Target sequence
gggcgccaagggactc

SEQ ID NO: 18 - protein, 7 aa, Simian virus 40
PKKKRKV

SEQ ID NO: 19 - protein, 15 aa, Xenopus sp.
KRPAATKKAGQAKKK